SEQUENCE LISTING (55 sequences)

SEQ ID NO: 1 - protein, 18 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  VARIANT 16: note = May be modified with a terminal amide or terminal methylamine
  VARIANT 17..18: note = This region may be absent or encompass a Glycine-Lysine amide, Lysine amide, Lysine-Lysine amide, or Glycine-Lysine amide where the lysine sidechain is modified with a methoxyethane, a Indium 111-1, 4, 7 - triazacyclononane - N, N ', N "- triacetic acid phenyl thiourea, a Gallium 67-1, 4, 7 - triazacyclononane - N, N ', N "- triacetic acid phenyl thiourea, a Gallium-68-1, 4, 7 - triazacyclononane - N, N ', N "- triacetic acid phenyl thiourea, a fluorine-18-aluminum-fluoride-1, 4, 7 - triazacyclononane - N, N ', N "- triacetic acid phenyl thiourea, a Copper 64-1, 4, 7 - triazacyclononane - N, N ', N "- triacetic acid phenyl thiourea, or a 1, 4, 7 - triazacyclononane - N, N ', N "- triacetic acid phenyl thiourea
FFFYYDVDVDVYFHCGXX

SEQ ID NO: 2 - protein, 16 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 16: note = Amidated residue
FFFYYDVDVDVYFHCG

SEQ ID NO: 3 - protein, 18 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 18: note = Amidated residue
FFFYYDVDVDVYFHCGGK

SEQ ID NO: 4 - protein, 18 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 18: note = Lysine-amide where the lysine sidechain is modified with a 1, 4, 7 - triazacyclononane - N, N ', N "- triacetic acid phenyl thiourea
FFFYYDVDVDVYFHCGGK

SEQ ID NO: 5 - protein, 18 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 18: note = Lysine-amide where the lysine sidechain is modified with a methoxyethane
FFFYYDVDVDVYFHCGGK

SEQ ID NO: 6 - protein, 18 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 18: note = Lysine-amide where the lysine sidechain is modified with a Gallium-68-1, 4, 7 - triazacyclononane - N, N ', N "- triacetic acid phenyl thiourea
FFFYYDVDVDVYFHCGGK

SEQ ID NO: 7 - protein, 18 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 18: note = Lysine-amide where the lysine sidechain is modified with a fluorine-18-aluminum-fluoride-1, 4, 7 - triazacyclononane - N, N ', N "- triacetic acid phenyl thiourea
FFFYYDVDVDVYFHCGGK

SEQ ID NO: 8 - protein, 16 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 16: note = Amidated residue
FFFYYDVDVDVYFHCG

SEQ ID NO: 9 - protein, 18 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 18: note = Amidated residue
FFFYYDVDVDVYFHCGGK

SEQ ID NO: 10 - protein, 18 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 18: note = Lysine-amide where the lysine sidechain is modified with a Gallium 67-1, 4, 7 - triazacyclononane - N, N ', N "- triacetic acid phenyl thiourea
FFFYYDVDVDVYFHCGGK

SEQ ID NO: 11 - protein, 18 aa, synthetic construct
  SITE 2: note = N-Methyl phenylalanine
  SITE 13: note = N-Methyl phenylalanine
  SITE 1..15: note = Side chain linked to N-terminus of residue in position 1
  SITE 18: note = Amidated residue
FFAYYDVDVDVYFHCGGK

SEQ ID NO: 12 - protein, 17 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 17: note = Amidated residue
FFFYADVDVDVYFHCGK

SEQ ID NO: 13 - protein, 18 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 18: note = Amidated residue
FFFYADVDVDVYFHCGKK

SEQ ID NO: 14 - protein, 17 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 17: note = Amidated residue
FFFYYDVDVDVYFHCGK

SEQ ID NO: 15 - protein, 18 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 18: note = Amidated residue
FFFYYDVDVDVYFHCGKK

SEQ ID NO: 16 - protein, 16 aa, synthetic construct
  SITE 1: note = Chloroacetylated residue
  SITE 2: note = N-Methyl alanine
  SITE 3: note = N-Methyl phenylalanine
  SITE 13: note = N-Methyl phenylalanine
  SITE 16: note = Amidated residue
FAFYYDVDVDVYFHCG

SEQ ID NO: 17 - protein, 18 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 18: note = Lysine-amide where the lysine sidechain is modified with a 1, 4, 7 - triazacyclononane - N, N ', N "- triacetic acid phenyl thiourea
FFFYYDVDADVYFHCGGK

SEQ ID NO: 18 - protein, 18 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 18: note = Lysine-amide where the lysine sidechain is modified with a Indium 111-1, 4, 7 - triazacyclononane - N, N ', N "- triacetic acid phenyl thiourea
FFFYYDVDVDVYFHCGGK

SEQ ID NO: 19 - protein, 18 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 18: note = Lysine-amide where the lysine sidechain is modified with a fluorine-18-aluminum-fluoride-1, 4, 7 - triazacyclononane - N, N ', N "- triacetic acid phenyl thiourea
FFFYYDVDADVYFHCGGK

SEQ ID NO: 20 - protein, 18 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 18: note = Lysine-amide where the lysine sidechain is modified with a Indium 111-1, 4, 7 - triazacyclononane - N, N ', N "- triacetic acid phenyl thiourea
FFFYYDVDVDVYFHCGGK

SEQ ID NO: 21 - protein, 18 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 18: note = Lysine-amide where the lysine sidechain is modified with a Gallium 67-1, 4, 7 - triazacyclononane - N, N ', N "- triacetic acid phenyl thiourea
FFFYYDVDVDVYFHCGGK

SEQ ID NO: 22 - protein, 16 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 16: note = Amidated residue
FFFYYDVDVDVYFACG

SEQ ID NO: 23 - protein, 16 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 16: note = Amidated residue
FFFYADVDVDVYFHCG

SEQ ID NO: 24 - protein, 16 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 16: note = Amidated residue
FFFYYDVDADVYFHCG

SEQ ID NO: 25 - protein, 16 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 16: note = Amidated residue
FFFYYDVDVDAYFHCG

SEQ ID NO: 26 - protein, 16 aa, synthetic construct
  SITE 2: note = N-Methyl phenylalanine
  SITE 13: note = N-Methyl phenylalanine
  SITE 1..15: note = Side chain linked to N-terminus of residue in position 1
  SITE 16: note = Amidated residue
FFAYYDVDVDVYFHCG

SEQ ID NO: 27 - protein, 16 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 16: note = Amidated residue
AFFYYDVDVDVYFHCG

SEQ ID NO: 28 - protein, 16 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 16: note = Amidated residue
FFFYYDADVDVYFHCG

SEQ ID NO: 29 - protein, 16 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 16: note = Amidated residue
FFFYYDVDVDVAFHCG

SEQ ID NO: 30 - protein, 16 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 16: note = Amidated residue
FFFYYDVAVDVYFHCG

SEQ ID NO: 31 - protein, 16 aa, synthetic construct
  SITE 2: note = N-Methyl phenylalanine
  SITE 3: note = N-Methyl phenylalanine
  SITE 13: note = N-Methyl phenylalanine
  SITE 1..15: note = Side chain linked to N-terminus of residue in position 1
  SITE 16: note = Amidated residue
FFFAYDVDVDVYFHCG

SEQ ID NO: 32 - protein, 16 aa, synthetic construct
  SITE 2: note = N-Methyl phenylalanine
  SITE 3: note = N-Methyl alanine
  SITE 13: note = N-Methyl phenylalanine
  SITE 1..15: note = Side chain linked to N-terminus of residue in position 1
  SITE 16: note = Amidated residue
FFAYYDVDVDVYFHCG

SEQ ID NO: 33 - protein, 16 aa, synthetic construct
  SITE 2: note = N-Methyl alanine
  SITE 3: note = N-Methyl phenylalanine
  SITE 13: note = N-Methyl phenylalanine
  SITE 1..15: note = Side chain linked to N-terminus of residue in position 1
  SITE 16: note = Amidated residue
FAFYYDVDVDVYFHCG

SEQ ID NO: 34 - protein, 18 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 18: note = Lysine-amide where the lysine sidechain is modified with a 1, 4, 7 - triazacyclononane - N, N ', N "- triacetic acid phenyl thiourea
FFFYYDVDVDVYFHCGGK

SEQ ID NO: 35 - protein, 18 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 18: note = Lysine-amide where the lysine sidechain is modified with a methoxyethane
FFFYYDVDVDVYFHCGGK

SEQ ID NO: 36 - protein, 18 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 18: note = Lysine-amide where the lysine sidechain is modified with a Gallium-68-1, 4, 7 - triazacyclononane - N, N ', N "- triacetic acid phenyl thiourea
FFFYYDVDVDVYFHCGGK

SEQ ID NO: 37 - protein, 18 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 18: note = Lysine-amide where the lysine sidechain is modified with a fluorine-18-aluminum-fluoride-1, 4, 7 - triazacyclononane - N, N ', N "- triacetic acid phenyl thiourea
FFFYYDVDVDVYFHCGGK

SEQ ID NO: 38 - protein, 18 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 18: note = Lysine-amide where the lysine sidechain is modified with a fluorine-18-aluminum-fluoride-1, 4, 7 - triazacyclononane - N, N ', N "- triacetic acid phenyl thiourea
FFFYYDVDADVYFHCGGK

SEQ ID NO: 39 - protein, 18 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 18: note = Lysine-amide where the lysine sidechain is modified with a 1, 4, 7 - triazacyclononane - N, N ', N "- triacetic acid phenyl thiourea
FFFYYDVDADVYFHCGGK

SEQ ID NO: 40 - protein, 18 aa, synthetic construct
  SITE 1: note = Chloroacetylated residue
  SITE 2: note = N-Methyl phenylalanine
  SITE 13: note = N-Methyl phenylalanine
  SITE 18: note = Amidated residue
FFAYYDVDVDVYFHCGGK

SEQ ID NO: 41 - protein, 17 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 17: note = Amidated residue
FFFYADVDVDVYFHCGK

SEQ ID NO: 42 - protein, 18 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 18: note = Amidated residue
FFFYADVDVDVYFHCGKK

SEQ ID NO: 43 - protein, 17 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 17: note = Amidated residue
FFFYYDVDVDVYFHCGK

SEQ ID NO: 44 - protein, 18 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 18: note = Amidated residue
FFFYYDVDVDVYFHCGKK

SEQ ID NO: 45 - protein, 16 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 16: note = Amidated residue
FFFYYDVDVDVYFACG

SEQ ID NO: 46 - protein, 16 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 16: note = Amidated residue
FFFYADVDVDVYFHCG

SEQ ID NO: 47 - protein, 16 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 16: note = Amidated residue
FFFYYDVDADVYFHCG

SEQ ID NO: 48 - protein, 16 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 16: note = Amidated residue
FFFYYDVDVDAYFHCG

SEQ ID NO: 49 - protein, 16 aa, synthetic construct
  SITE 1: note = Chloroacetylated residue
  SITE 2: note = N-Methyl phenylalanine
  SITE 13: note = N-Methyl phenylalanine
  SITE 16: note = Amidated residue
FFAYYDVDVDVYFHCG

SEQ ID NO: 50 - protein, 16 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 16: note = Amidated residue
AFFYYDVDVDVYFHCG

SEQ ID NO: 51 - protein, 16 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 16: note = Amidated residue
FFFYYDADVDVYFHCG

SEQ ID NO: 52 - protein, 16 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 16: note = Amidated residue
FFFYYDVDVDVAFHCG

SEQ ID NO: 53 - protein, 16 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 16: note = Amidated residue
FFFYYDVAVDVYFHCG

SEQ ID NO: 54 - protein, 16 aa, synthetic construct
  SITE 1: note = Chloroacetylated residue
  SITE 2: note = N-Methyl phenylalanine
  SITE 3: note = N-Methyl phenylalanine
  SITE 13: note = N-Methyl phenylalanine
  SITE 16: note = Amidated residue
FFFAYDVDVDVYFHCG

SEQ ID NO: 55 - protein, 16 aa, synthetic construct
  SITE 1: note = Chloroacetylated residue
  SITE 2: note = N-Methyl phenylalanine
  SITE 3: note = N-Methyl alanine
  SITE 13: note = N-Methyl phenylalanine
  SITE 16: note = Amidated residue
FFAYYDVDVDVYFHCG

(19) United States
(12) Patent Application Publication
(10) Pub. No.: US 2026/0297134 A1
(43) Pub. Date: Oct. 1, 2026

(54) CYCLIC PEPTIDES AS PET IMAGING AGENTS OF GRANZYME B

(71) Applicant: Merck Sharp & Dohme LLC, Rahway, NJ (US)

(72) Inventors: Idriss Bennacef, Ambler, PA (US); Tapan K. Nayak, Lansdale, PA (US); Jeffrey William Schubert, North Wales, PA (US); William D. Shipe, Chalfont, PA (US); David M. Tellers, Lansdale, PA (US); Federica Orvieto, Pomezia (IT); Valerio Piacenti, Pomezia (IT)

(73) Assignee: Merck Sharp & Dohme LLC, Rahway, NJ (US)

(21) Appl. No.: 19/479,468

(22) PCT Filed: Apr. 29, 2024
(86) PCT No.: PCT/US2024/026735

§ 371 Date: Oct. 28, 2025

Related U.S. Application Data
(60) Provisional application No. 63/499,816, filed on May 3, 2023.

(51) Int. Cl. C07K 7/64; A61K 38/00; A61K 51/08

(52) U.S. Cl. CPC C07K 7/64; A61K 51/08; A61K 38/00

(57) ABSTRACT

Disclosed are cyclic peptides, their salts, pharmaceutical compositions comprising them, diagnostic and therapeutic uses and processes for making such compounds, which bind to granzyme B and may be suitable for imaging granzyme B. Further provided are compounds useful as a radiotracer for positron emission tomography (PET) and/or single photon emission tomography (SPECT) imaging. The use of the compounds as imaging agents of granzyme B is further disclosed.

### CROSS-REFERENCE TO RELATED APPLICATIONS

This application claims the benefit of priority of U.S. Provisional Application No. 63/499,816, filed May 3, 2023, the disclosure of which is incorporated herein by its entirety.

### REFERENCE TO SEQUENCE LISTING SUBMITTED ELECTRONICALLY

This instant application contains a Sequence listing which has been submitted electronically in XML format and is hereby incorporated by reference in its entirety. The XML file, created on Nov. 7, 2023, is named 25689_WO_PCT_SL.XML and is 154,816 bytes in size.

### BACKGROUND

Granzyme B (GzmB) is a serine protease secreted by cytotoxic T lymphocytes (CTLs) and natural killer (NK) cells. GzmB-induced cell death has been traditionally viewed as a primary mechanism used by CTLs and NK cells to eliminate harmful target cells including allogeneic, virally infected, and tumor cells. See US2019/0224348; Larimer, B.; et al. Cancer Res 77, 2017, 2318-2327 and Larimer. B.; et al. U. Clin Cancer Res 25, 2019, 1196-1205. Measurement of GzmB can serve as a readout for the functional status of CTLs and NKs and is of interest in cancer immunotherapy and autoimmunity. Presently disclosed are cyclic peptides that bind human granzyme B with high affinity. These peptides can be conjugated to a chelator and radioactive metal for use in PET imaging of granzyme B.

### SUMMARY OF THE DISCLOSURE

Provided are novel cyclic peptides, their salts, pharmaceutical compositions comprising them, diagnostic and therapeutic uses and processes for making such compounds, which may be suitable for imaging granzyme B. Further provided are compounds useful as a radiotracer for positron emission tomography (PET) and/or single photon emission tomography (SPECT) imaging. This disclosure further relates to use of the compounds as imaging agents of granzyme B.

### BRIEF DESCRIPTION OF THE FIGURES

FIG. 1: Shows representative 3D PET/CT images of mice injected with ¹⁸F-labeled anti-granzyme-B peptide 6a and corresponding granzyme-B IHC staining from lung tissue collected after the final PET scan.

FIG. 2: (A) Shows Representative PET/CT images at different time-points from male rhesus monkey injected with ¹⁸F-labeled anti-granzyme-B peptide 6a. (B) Shows corresponding standardized uptake value (SUV) mean in tissues of interest derived from male rhesus monkey injected with 18F-labeled anti-granzyme-B peptide 6a.

### DETAILED DESCRIPTION OF THE DISCLOSURE

Described are compounds of Formula I or a pharmaceutically acceptable salt thereof, which are capable of binding granzyme B. In some embodiments, the compound of Formula I, or a pharmaceutically acceptable salt thereof is an irreversible binder of granzyme B. In some embodiments, the compound of Formula I, or a pharmaceutically acceptable salt thereof is an inhibitor of granzyme B. Still, in some embodiments, the compounds of Formula I, or a pharmaceutically acceptable salt thereof, contain one or more imaging agents. In some embodiments, the compound of Formula I, or a pharmaceutically acceptable salt thereof is a polypeptide that binds granzyme B.

Provided are novel macrocyclic peptides of Formula I:

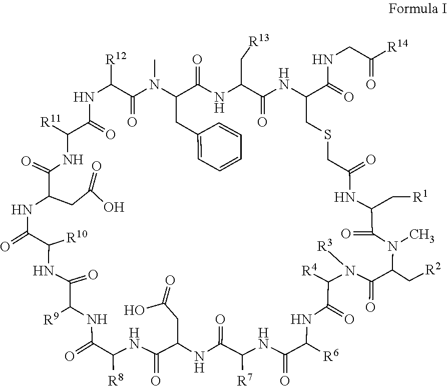

and pharmaceutically acceptable salts thereof, wherein:
R¹ and R² are each independently selected from hydrogen, C₁₋₆ alkyl and C₆₋₁₀ aryl, said aryl optionally substituted with 1 to 3 groups selected from C₁₋₆ alkyl, halogen, and hydroxyl;
R³ is hydrogen or CH₃;
R⁴, R⁶, R⁷, and R¹² are each independently selected from C₁₋₆ alkyl, and —CH₂-aryl optionally substituted with 1 to 3 substituents selected from hydroxy, C₁₋₆ alkyl and halogen,
R⁸, R⁹, R¹⁰, and R¹¹ are each independently hydrogen or C₁₋₆ alkyl, said alkyl optionally substituted with 1 to 3 groups selected from R^{a};
R^{a} is selected from hydroxyl, —COOH, —NH₂, —NHC(═NH₂)NH₂ and C(O)NH₂;
R¹³ is selected from C₃₋₁₀heteroaryl and hydrogen, said heteroaryl optionally substituted with 1 to 3 groups selected from OH, C₁₋₆ alkyl and halogen;
R¹⁴ is selected from —NH₂—CH₂NH₂,

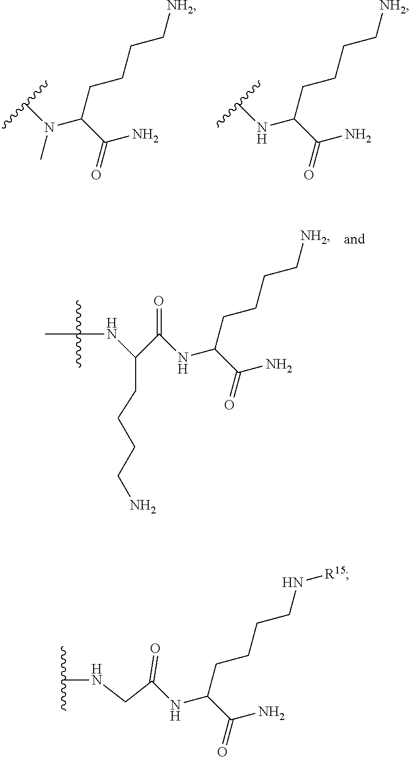

wherein R¹⁵ is selected from
(i)

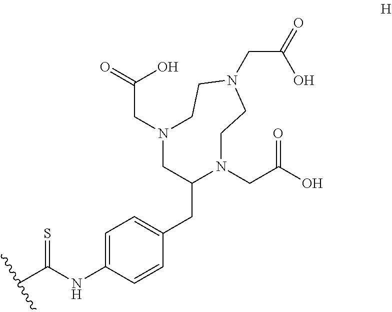 [SMILES: *C(=S)Nc1ccc(CC2CN(CC(=O)O)CCN(CC(=O)O)CCN2CC(=O)O)cc1]

(ii)
(iii) R^{0a}C(O)— wherein R^{0a} is C₁₋₃ alkyl, or a polyethylene glycol polymer;
(iv) a chelating moiety, optionally having a positron emitting isotope as an imaging agent.

An embodiment of Formula I of this disclosure is realized by a compound of structural Formula I′, or a pharmaceutically acceptable salt thereof:

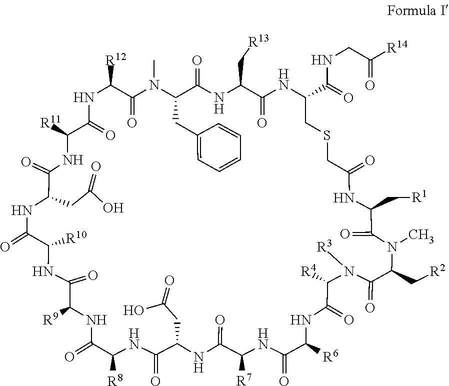

wherein R¹, R², R³, R⁴, and R⁶ through R¹⁴ are as described herein. An embodiment of this aspect of the invention is realized when R¹⁴ is selected from —NH₂, —CH₂NH₂,

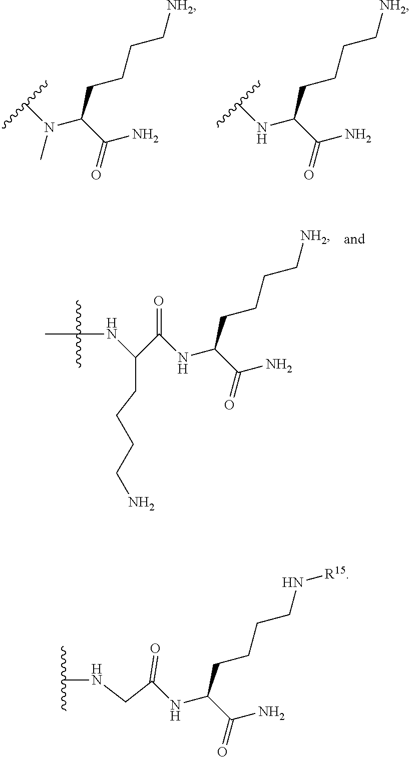

An embodiment of this disclosure is realized when R¹ is C₁₋₆ alkyl. A subembodiment of this aspect of the disclosure is realized when R¹ is methyl, or —CH(CH₃)₂.

An embodiment of this disclosure is realized when R¹ is hydrogen.

Another embodiment of this disclosure is realized when R¹ is C₆₋₁₀ aryl, optionally substituted with 1 to 3 groups selected from C₁₋₆ alkyl, halogen, and hydroxyl. A further subembodiment is realized R¹ is unsubstituted phenyl. A subembodiment of this aspect of the disclosure is realized when R¹ is phenyl, optionally substituted with 1 to 3 groups selected from C₁₋₆ alkyl, fluorine and hydroxyl. A further subembodiment is realized R¹ is phenyl, optionally para-substituted with hydroxyl, methyl, or fluoro.

An embodiment of this disclosure is realized when R¹ is hydrogen.

Another embodiment of this disclosure is realized when R¹ is selected from hydrogen, methyl, —CH(CH₃)₂, or phenyl optionally substituted with 1 to 3 groups selected from C₁₋₆ alkyl, fluorine and hydroxyl.

An embodiment of this disclosure is realized when R² is C₁₋₆ alkyl. A subembodiment of this aspect of the disclosure is realized when R² is methyl, or —CH(CH₃)₂.

An embodiment of this disclosure is realized when R² is hydrogen.

Another embodiment of this disclosure is realized when R² is C₆₋₁₀ aryl, optionally substituted with 1 to 3 groups selected from C₁₋₆ alkyl, halogen, and hydroxyl. A further subembodiment is realized R² is unsubstituted phenyl. A subembodiment of this aspect of the disclosure is realized when R² is phenyl, optionally substituted with 1 to 3 groups selected from C₁₋₆ alkyl, fluorine and hydroxyl. A further subembodiment is realized R² is phenyl, optionally para-substituted with hydroxyl, methyl, or fluoro.

Another embodiment of this disclosure is realized when R² is selected from hydrogen, methyl. —CH(CH₃)₂, or phenyl optionally substituted with 1 to 3 groups selected from C₁₋₆ alkyl, fluorine and hydroxyl.

An embodiment of this disclosure is realized when R³ is hydrogen.

An embodiment of this disclosure is realized when R³ is CH₃.

An embodiment of this disclosure is realized when R⁴ is C₁₋₆ alkyl. A subembodiment of this aspect of the disclosure is realized when R⁴ is methyl, or —CH(CH₃)₂.

Another embodiment of this disclosure is realized when R⁴ is C₆₋₁₀ aryl, optionally substituted with 1 to 3 groups selected from C₁₋₆ alkyl, halogen, and hydroxyl. A further subembodiment is realized R⁴ is unsubstituted phenyl. A subembodiment of this aspect of the disclosure is realized when R⁴ is phenyl, optionally substituted with 1 to 3 groups selected from C₁₋₆ alkyl, fluorine and hydroxyl. A further subembodiment is realized R⁴ is phenyl, optionally para-substituted with hydroxyl, methyl, or fluoro.

An embodiment of this disclosure is realized when R⁴ is selected from methyl and phenyl, optionally substituted with 1 to 3 groups selected from C₁₋₆ alkyl, fluorine and hydroxyl.

Another embodiment of this disclosure is realized when one of R² and R⁴ is C₁₋₆ alkyl and the other is C₆₋₁₀ aryl or CH₂C₆₋₁₀ aryl, said aryl optionally substituted with 1 to 3 groups selected from C₁₋₆ alkyl, halogen, and hydroxyl. A subembodiment of this aspect of the invention is realized when R² and R⁴ is C₆₋₁₀ aryl or CH₂C₆₋₁₀ aryl, said aryl optionally substituted with 1 to 3 groups selected from C₁₋₆ alkyl, halogen, and hydroxyl. Another subembodiment of this aspect of the invention is realized when R² and R⁴ is phenyl or CH₂phenyl, optionally substituted with 1 to 3 groups selected from C₁₋₆ alkyl, fluorine and hydroxyl. Still another subembodiment of this aspect of the invention is realized when R² and R⁴ is phenyl or CH₂phenyl, said phenyl optionally substituted with 1 to 3 groups of OH. Yet another subembodiment of this aspect of the invention is realized when each of R² is unsubstituted phenyl and R⁴ is CH₂phenyl unsubstituted or substituted with 1 to 3 groups of OH.

An embodiment of this disclosure is realized when R⁶ is C₁₋₆ alkyl. A subembodiment of this aspect of the disclosure is realized when R⁶ is methyl, or —CH(CH₃)₂.

Another embodiment of this disclosure is realized when R⁶ is CH₂Cl₆₋₁₀ aryl, optionally substituted with 1 to 3 groups selected from C₁₋₆ alkyl, halogen, and hydroxyl. A further subembodiment is realized when R⁶ is unsubstituted CH₂Cphenyl. A subembodiment of this aspect of the disclosure is realized when R⁶ is CH₂Cphenyl, optionally substituted with 1 to 3 groups selected from C₁₋₆ alkyl, fluorine and hydroxyl. A further subembodiment is realized when R⁶ is CH₂Cphenyl, optionally para-substituted with hydroxyl, methyl, or fluoro. A further subembodiment is realized when R⁶ is CH₂Cphenyl, substituted with hydroxyl, methyl, or fluoro. A further subembodiment is realized when R⁶ is CH₂Cphenyl, substituted with hydroxyl.

An embodiment of this disclosure is realized when R⁷ is C₁₋₆ alkyl. A subembodiment of this aspect of the disclosure is realized when R⁷ is methyl, or —CH(CH₃)₂.

Another embodiment of this disclosure is realized when R⁷ is CH₂C₆₋₁₀ aryl, optionally substituted with 1 to 3 groups selected from C₁₋₆ alkyl, halogen, and hydroxyl. A further subembodiment is realized R⁷ is unsubstituted CH₂Cphenyl. A subembodiment of this aspect of the disclosure is realized when R⁷ is CH₂Cphenyl, optionally substituted with 1 to 3 groups selected from C₁₋₆ alkyl, fluorine and hydroxyl. A further subembodiment is realized when R⁷ is CH₂Cphenyl, optionally para-substituted with hydroxyl, methyl, or fluoro. A further subembodiment is realized when R⁷ is CH₂Cphenyl, substituted with hydroxyl, methyl, or fluoro. A further subembodiment is realized when R⁷ is CH₂Cphenyl substituted with hydroxyl.

An embodiment of this disclosure is realized when R¹² is C₁₋₆ alkyl. A subembodiment of this aspect of the disclosure is realized when R¹² is methyl, or —CH(CH₃)₂.

Another embodiment of this disclosure is realized when R¹² is CH₂C₆₋₁₀ aryl, optionally substituted with 1 to 3 groups selected from C₁₋₆ alkyl, halogen, and hydroxyl. A further embodiment of this disclosure is realized when R¹² is unsubstituted CH₂Cphenyl. A subembodiment of this aspect of the disclosure is realized when R¹² is CH₂Cphenyl, optionally substituted with 1 to 3 groups selected from C₁₋₆ alkyl, fluorine and hydroxyl. A further subembodiment is realized when R¹² is CH₂Cphenyl, optionally para-substituted with hydroxyl, methyl, or fluoro. A further subembodiment is realized when R¹² is CH₂Cphenyl, substituted with hydroxyl, methyl, or fluoro. A further subembodiment is realized when R¹² is CH₂Cphenyl substituted with hydroxyl. Still another subembodiment is realized R¹² is selected from CH₃, or CH₂Cphenyl, optionally substituted with 1 to 3 groups selected from C₁₋₆ alkyl, fluorine and hydroxyl.

Another embodiment of the disclosure is realized when R⁴, R⁶, R⁷, and R¹² are all CH₂Cphenyl optionally substituted with 1 to 3 substituents selected from hydroxyl, C₁₋₆ alkyl and halogen. A subembodiment of this aspect of the disclosure is realized when at least one of the CH₂Cphenyl groups of R⁴. R⁶, R⁷, and R¹² is substituted. Another subembodiment of this aspect of the disclosure is realized when at least two of the phenyl groups of R⁴, R⁶, R⁷, and R¹² are substituted. Another subembodiment of this aspect of the disclosure is realized when three of the phenyl groups of R⁴, R⁶, R⁷, and R¹² are substituted. Another subembodiment of this aspect of the disclosure is realized when one of the phenyl groups of R⁴, R⁶, R⁷, and R¹² is unsubstituted. Another subembodiment of this aspect of the disclosure is realized when R⁴ is unsubstituted CH₂Cphenyl and R⁶, R⁷, and R¹² are each CH₂Cphenyl substituted with 1 to 3 substituents selected from hydroxyl, C₁₋₆ alkyl and halogen. Another subembodiment of this aspect of the disclosure is realized when R⁴ is unsubstituted CH₂Cphenyl and R⁶, R⁷, and R¹² are each CH₂Cphenyl optionally substituted with 1 to 3 hydroxyl groups. Another subembodiment of this aspect of the disclosure is realized when R⁴ is unsubstituted CH₂Cphenyl and R⁶, R⁷, and R¹² are each CH₂Cphenyl optionally substituted with 1 hydroxyl group.

Another embodiment of the disclosure is realized when R⁸, R⁹, R¹⁰, and R¹¹ are each C₁₋₆ alkyl independently selected from methyl, ethyl, propyl, isopropyl, butyl, pentyl, and hexyl, said methyl, ethyl, propyl, isopropyl, butyl, pentyl, and hexyl optionally substituted with 1 to 3 groups selected from R^{a}. A further embodiment of this disclosure is realized when R⁸, R¹⁰, and R¹¹ are each optionally substituted isopropyl. A further embodiment of this disclosure is realized when R⁸, R¹⁰, and R^{11′} are each unsubstituted isopropyl. A further embodiment of this disclosure is realized when R⁹ is a methylene substituted with 1 to 3 groups selected from R. A further embodiment of this disclosure is realized when R⁹ is a methylene substituted with C(O)OH. A further embodiment of this disclosure is realized when R⁸, R¹⁰, and R¹¹ are each unsubstituted isopropyl and R⁹ is a methylene substituted with C(O)OH.

An embodiment of this disclosure is realized when R¹³ is hydrogen.

An embodiment of this disclosure is realized when R¹³ is C₃₋₁₀heteroaryl optionally substituted with 1 to 3 groups selected from OH, C₁₋₆ alkyl and halogen. A subembodiment this disclosure is realized when R¹³ is -imidazolyl, unsubstituted or substituted with fluoro or methyl.

Another embodiment of the disclosure is realized when R¹⁴ is NH₂, or CH₂NH₂.

Another embodiment of the disclosure is realized when R¹⁴ is

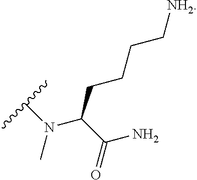 [SMILES: *N(C)[C@@H](CCCCN)C(N)=O]

Another embodiment of the disclosure is realized when R¹⁴ is

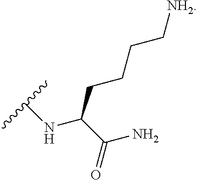 [SMILES: *N[C@@H](CCCCN)C(N)=O]

Another embodiment of the disclosure is realized when R¹⁴ is

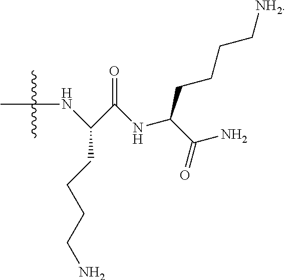 [SMILES: *N[C@@H](CCCCN)C(=O)N[C@@H](CCCCN)C(N)=O]

Another embodiment of the disclosure is realized when R¹⁴ is

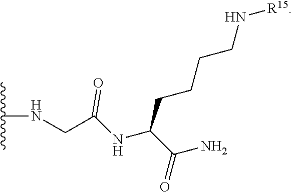

Another embodiment of the disclosure is realized when R¹⁵ is hydrogen. Another embodiment of the disclosure is realized when R¹⁵ is R^{0a}C(O)— wherein R^{0a} is C₁₋₃ alkyl, or a polyethylene glycol polymer selected from PEG 1-24.

Another embodiment of the disclosure is realized when R¹⁵ is a chelating moiety selected from desferrioxamine (DFO): 1, 4, 7, 10-tetraacetic acid (DOTA); diethylenetriaminepenaacetic acid (DTPA): ethylenediaminetetraacetic acid (EDTA); (1, 4, 7, 10-Tetraazacyclododecane-1, 4, 7, 10-tetra(methylene phosphonic) acid (DOTP); (1R, 4R, 7R, 10R)-α′α″α′″-tetramethyl-1, 4, 7, 10-tetraazacyclododecane-1, 4, 7, 10-tetraacetic acid (DOTMA): 1, 4, 8, 11-Tetraazacyclotetradecane-1, 4, 8, 11-tetraacetic acid (TETA); H4octapa, H6phospa, H2dedpa, H5decapa, H2azapa; HOPO; DO2A; 1, 4, 7, 10-Tetrakis(carbamoylmethyl)-1, 4, 7, 10-tetraazacyclododecane (DOTAM); 1, 4, 7-triazacyclononane-N, N′,N″-triacetic acid (NOTA); 1, 4, 8, 11-tetraazabicyclo[6.6.2]hexadecane-4, 11-dicetic acid (CB TE2A); 1, 4, 7, 10-Tetraazacyclododecane (Cyclen): 1, 4, 8, 11-tetraazacyclotetradecane (Cyclam), octadentate chelators, hexadentate chelators, phosphonate-based chelators, macrocyclic chelators, chelators comprising macrocyclic terephthalamide ligands, bifunctional chelators, fusarinine C and fusarinine C derivative chelators, triacetvlfusarinine C (TAFC), ferrioxamine E (FOXE), ferrioxamine B (FOXB), and ferrichrome A (FCHA), said chelating agent optionally having a positron emitting isotope as an imaging agent. A subembodiment is realized when the chelating agent is desferrioxamine (DFO), optionally having a positron emitting isotope. A subembodiment is realized when the chelating agent is 1, 4, 7, 10-tetraacetic acid (DOTA), optionally having a positron emitting isotope. A subembodiment is realized when the chelating agent is diethylenetriaminepenaacetic acid (DTPA), optionally having a positron emitting isotope. A subembodiment is realized when the chelating agent is ethylenediaminetetraacetic acid (EDTA), optionally having a positron emitting isotope. A subembodiment is realized when the chelating agent is (1, 4, 7, 10-Tetraazacyclododecane-1, 4, 7, 10-tetra(methylene phosphonic) acid (DOTP), optionally having a positron emitting isotope. A subembodiment of is realized when the chelating agent is (1R, 4R, 7R, 10R)-α′α″α′″-tetramethyl-1, 4, 7, 10-tetraazacyclododecane-1, 4, 7, 10-tetraacetic acid (DOTMA), optionally having a positron emitting isotope. A subembodiment is realized when the chelating agent is 1, 4, 8, 11-Tetraazacyclotetradecane-1, 4, 8, 11-tetraacetic acid (TETA), optionally having a positron emitting isotope. A subembodiment is realized when the chelating agent is selected from 6,6′-((ethane-1,2-diylbis((carboxymethyl)azanediyl))bis(methylene))dipicolinic acid (H4octapa), 6,6′-(2,3-bis((phosphonomethyl)amino)butane-1,4-diyl)dipicolinic acid (H6phospa). 6,6′-((ethane-1,2-diylbis(azanediyl))bis(methylene))dipicolinic acid (H2dedpa), 6,6′-(((((carboxymethyl)azanediyl)bis(ethane-2,1-diyl))bis((carboxymethyl)azanediyl))bis(methylene))dipicolinic acid (H5decapa), and 6,6′-(2,3-bis(((1-benzyl-1H-1,2,3-triazol-4-yl)methyl)amino)butane-1,4-diyl)dipicolinic acid (H2azapa), said group optionally having a positron emitting isotope. A subembodiment is realized when the chelating agent is N,N′-(butane-1,4-diyl)bis(1-hydroxy-N-(3-(1-hydroxy-6-oxo-1,6-dihydropyridine-2-carboxamido)propyl)-6-oxo-1,6-dihydropyridine-2-carboxamide) (HOPO), optionally having a positron emitting isotope. A subembodiment is realized when the chelating agent is 1,4,7,10-Tetraazacyclododecane-1,7-diacetic acid (DO2A), optionally having a positron emitting isotope. A subembodiment is realized when the chelating agent is 1, 4, 7, 10-Tetrakis(carbamoylmethyl)-1, 4, 7, 10-tetraazacyclododecane (DOTAM), optionally having a positron emitting isotope. A subembodiment is realized when the chelating agent is 1, 4, 7-triazacyclononane-N, N′,N″-triacetic acid (NOTA), optionally having a positron emitting isotope. A subembodiment is realized when the chelating agent is 1, 4, 8, 11-tetraazabicyclo[6.6.2]hexadecane-4, 11-dicetic acid (CB TE2A), optionally having a positron emitting isotope. A subembodiment is realized when the chelating agent is 1, 4, 7, 10-Tetraazacyclododecane (Cyclen), optionally having a positron emitting isotope. A subembodiment is realized when the chelating agent is 1, 4, 8, 11-tetraazacyclotetradecane (Cyclam), optionally having a positron emitting isotope. A subembodiment is realized when the chelating agent is selected from octadentate chelators, hexadentate chelators, phosphonate-based chelators, macrocyclic chelators, chelators comprising macrocyclic terephthalamide ligands, bifunctional chelators, fusarinine C and fusarinine C derivative chelators, said group optionally having a positron emitting isotope. A subembodiment is realized when the chelating agent is triacetylfusarinine C (TAFC), optionally having a positron emitting isotope. A subembodiment is realized when the chelating agent is ferrioxamine E (FOXE), optionally having a positron emitting isotope. A subembodiment is realized when the chelating agent is ferrioxamine B (FOXB), optionally having a positron emitting isotope. A subembodiment is realized when the chelating agent is ferrichrome A (FCHA), optionally having a positron emitting isotope. Another embodiment is realized when R14 is a chelating agent optionally having a positron emitting isotope as an imaging agent selected from DOTA, NOTA, RESCA 68Ga, 67Ga, 64Cu, and Al18F. Another subembodiment is realized when the chelating agent is selected from 177Lu, 90Y. and 111In.

Another embodiment of the disclosure is realized when R¹⁵ is

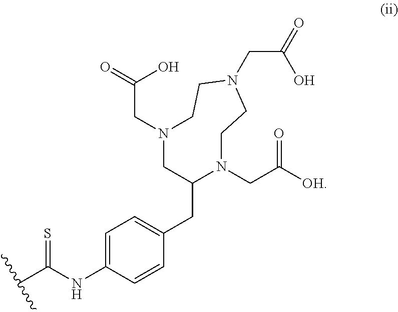 [SMILES: *C(=S)Nc1ccc(CC2CN(CC(=O)O)CCN(CC(=O)O)CCN2CC(=O)O)cc1]

A subembodiment of this aspect of the disclosure is realized when the structure of (ii) optionally contains a positron emitting isotope. Another subembodiment is realized when the positron emitting isotope is selected from ⁶⁸Ga, ¹⁸F (in Al¹⁸F), and ⁶⁴Cu.

Another embodiment of the disclosure of Formula I is realized by structural Formula Ia

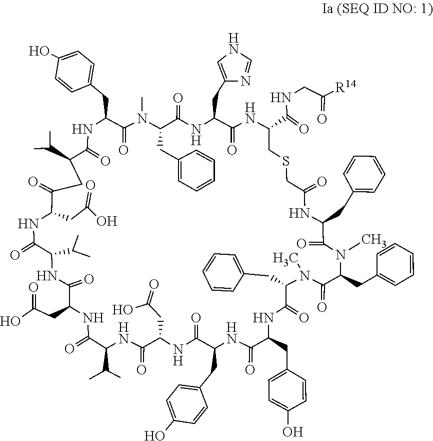

wherein R¹⁴ is as described herein.

A subembodiment of the disclosure of Formula Ia is realized when it contains 1 or more positron emitting imaging agents.

An embodiment of the disclosure is realized when the compounds of Formula I, Formula I′ and Formula Ia contain one or more imaging agents. In another embodiment, the imaging agents are selected from the group consisting of a paramagnetic ion, an x-ray imaging agent, a fluorophore, and a radioisotope. Many appropriate imaging agents are known in the art, as are methods for their attachment to antibodies (see, for e.g., U.S. Pat. Nos. 5,021,236; 4,938,948; and 4,472,509, the disclosure of each of which is incorporated herein by reference in its entirety). Radioactively labeled compounds of Formula I, Formula I′ and Formula Ia or a pharmaceutically acceptable salt thereof provided herein may be prepared according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. In a further example, compounds of Formula I, Formula I′ and Formula Ia or a pharmaceutically acceptable salt thereof, provided herein may be labeled with ⁶⁸Ga by radiometalation of a bifunctional chelator provided herein (e.g., NOTA, DOTA, or NODAGA) or a similar derivative thereof. Synthetic methods for incorporating radioisotopes into organic compounds are well known in the art, and one of ordinary skill in the art will readily recognize other methods applicable for the compounds provided herein.

In another embodiment, the imaging agent comprises 1, 2, or 3 imaging agents selected from the group consisting of a paramagnetic ion, an x-ray imaging agent, a fluorophore, and a radioisotope. In another embodiment, Formula I, Formula I′ and Formula Ia comprise one imaging agent. In another embodiment Formula I, Formula I′ and Formula Ia comprise two imaging agents. In another embodiment. Formula I, Formula I′ and Formula La comprise three imaging agents. In another embodiment the compound of Formula I, Formula I′ and Formula Ia comprise one or more imaging agents which can include one or more independently selected paramagnetic ions.

A subembodiment of this aspect of the disclosure is realized when each of the paramagnetic ions are independently selected from the group consisting of fluoride, chromium (III), manganese (IL), iron (III), iron (IL), cobalt (II), nickel (LI), copper (II), neodymium (III), samarium (III), ytterbium (III), gado-linium (III), vanadium (II), terbium (III), dysprosium (III), holmium (III), and erbium (III). Another subembodiment of this aspect of the disclosure is realized when the compound of Formula I, Formula I′ or Formula Ia comprise 1, 2, or 3 independently selected paramagnetic ions. Another subembodiment of this aspect of the disclosure is realized when the one or more independently paramagnetic ions are independently directly or indirectly (e.g., through a chelator) bound to the compounds provided herein.

Another embodiment of this aspect of the disclosure is realized when the compound of Formula I, Formula I′ aor Formula Ia comprises one or more imaging agents which are independently selected x-ray imaging agents. A subembodiment of this aspect of the disclosure is realized when each of the x-ray imaging agents are independently selected from the group consisting of lanthanum (III), gold (III), lead (II), bismuth(III), and iodinated x-ray imaging agents (e.g., diatrizoate, ioxaglate, metrizoate, iopamidol, iohexol, ioxilan, iopromide, iodixanol, and ioversol).

Another embodiment of this disclosure is realized when the imaging agent is a fluorophore. A subembodiment of this aspect of the disclosure is realized when A is a fluorophore selected from the group consisting of Alexa 350, Alexa 430, AMCA, BODIPY 630/650, BODIPY 650/665, BODIPY-FL, BODPY-R6G, 13BODLPY-TMR, BODLPY-TRX, cascade blue. Cy3, Cy5, 6-FAM, fluorescein isothiocyanate, HEX, 6-JOE, Oregon green 488, Oregon green 500, Oregon green 514, a quantum dot, pacific blue, REG, rhodamine green, rhodamine red, renographin, ROX, TAMRA, TET, tetramethyl-rhodamine, Texas Red, AF 350, 405, AF532, AF488, AF647, AF680, AF750, Cy5, Cy5.5, Cy7, indocyanine green (ICG), green fluorescent protein (GFP), red fluorescent protein (RFP), dsRED, and IRdye 800.

Another embodiment of this aspect of the disclosure is realized when the compound of Formula I, Formula I′ or Formula Ia comprises one or more imaging agents which include one or more independently selected radioisotopes. A subembodiment of this aspect of the disclosure is realized when the radioisotopes provided herein are useful as imaging agents in one or more of the methods provided herein. A subembodiment of this aspect of the disclosure is realized when one or more of the radioistopes provided herein may also be useful in one or more therapeutic applications, (e.g., when administered to a subject in a therapeutically effective amount). ¹³¹I and ⁶⁴Cu, for example, may be useful as imaging agents (e.g., as non-toxic and/or non-therapeutic radioisotopes) when administered to the subject at low concentrations (e.g., 5 mCi) and may also be useful as therapeutic agents (i.e., as toxic radioisotopes and/or therapeutic radioisotopes) when administered to the subject at a higher concentration. A subembodiment of this aspect of the disclosure is realized when each of the radioisotopes are independently selected from the group consisting of ³H, 11C, ¹⁴C, ¹⁸F, ³²P ³⁵S, ³⁶Cl, ⁵¹Cr ⁵²Fe, ⁵⁷Co, ⁵⁸Co, ⁵⁹Fe, ⁶⁴Cu, ⁶⁷Cu, ⁶⁷Ga, ⁶⁸Ga, ⁷⁵Se, ⁷⁶Br, ⁷⁷Br, ⁸⁹Zr, ⁹⁰Y, ^{99m}Tc ¹¹¹In ¹²³I ¹²⁴I ¹²⁵I ¹³¹I ¹⁵²Eu ¹⁵³Sm ¹⁶⁶Ho, ¹⁷⁷Lu, ¹⁸⁶Re, ¹⁸⁸Re, ²⁰¹Tl, ²⁰³Pb ²¹²Pb, ²¹⁰At, ²¹¹At, ²¹²Bi, ²¹³Bi, and ²²⁵Ac. A subembodiment of this aspect of the disclosure is realized when one or more independently radioisotopes are independently directly or indirectly (e.g., through a chelator) bound to the compounds of Formula I, Formula I′ and Formula Ia.

Another embodiment of this disclosure is realized when the compound of Formula I, Formula I′ or Formula Ia comprises one or more imaging agents selected from PET (positron emission tomography), SPECT (single-photon emission computed tomography), and computed tomography imaging agent. A subembodiment of this aspect of the disclosure is realized when the imaging agent is PET. Another subembodiment of this aspect of the disclosure is realized when the imaging agent is SPECT. Another subembodiment of this aspect of the disclosure is realized when the imaging agent is computed tomography imaging agent. Another subembodiment of this aspect of the disclosure is realized when the imaging agent is a radioisotopic computed tomography imaging agent. Another subembodiment of the disclosure is realized when the imaging agent is PET or SPECT comprising one or more radioisotopes selected from ¹¹C, ¹⁸F, ⁶⁴Cu, ⁶⁷Ga, ⁶⁸Ga, ⁷⁶Br, ⁷⁷Br, ⁸⁹Zr, ¹¹¹In, 123, 1241 ¹⁸⁶Re, ¹⁸⁸Re, and 20Tl. In further aspects of the disclosure the imaging agent is a PET or SPECT comprising ⁶⁷Ga or ⁶⁸Ga. In further aspects, non-limiting radioisotopes that form stable complexes with a chelating moiety and have physical half-lives suitable for PET imaging purposes are selected from ⁸⁹Zr, ⁶⁸Ga, ⁶⁴Cu, ⁴⁴Sc, and ⁸⁶Y. In further aspects, non-limiting radioisotopes that directly bond with peptide, including, but not limited to. ⁷⁶Br and ¹²⁴I. Still in further aspects, non-limiting radioisotopes that are introduced via prosthetic group are, for example, ¹⁸F.

Another embodiment of the disclosure is realized when the compound of Formula I, Formula I′ and Formula La, or a pharmaceutically acceptable salt thereof is linked to one or more imaging agents through a linking group. A subembodiment of this aspect of the disclosure is realized when the linking group comprises one or more amino acid residues. A subembodiment of this aspect of the disclosure is realized when there are about 1 to about 100, about 1 to about 80, about 1 to about 60, about 1 to about 40, about 1 to about 20, about 1 to about 10, about 1 to about 5, about 5 to about 100, about 5 to about 80, about 5 to about 60, about 5 to about 40, about 5 to about 20, about 5 to about 10, about 10 to about 100, about 10 to about 80, about 10 to about 60, about 10 to about 40, about 10 to about 20, about 20 to about 100, about 20 to about 80, about 20 to about 60, about 20 to about 40, about 40 to about 100, about 40 to about 80, about 40 to about 60, about 60 to about 100, about 60 to about 80, or about 80 to about 100 amino acid residues.

Another embodiment of this aspect of the disclosure is realized when the linking group comprises one or more alkylene groups, one or more amine groups, one or more amide groups, one or more alkylenoxy groups, one or more thiol groups, one or more carbohydrate groups, or any combination thereof. A subembodiment of this aspect of the disclosure is realized when the linking group comprises one or more C₁₋₅₀ alkylene groups, one or more amine groups, one or more amide groups, one or more C₁₋₅₀ alkylenoxy groups, one or more C₁₋₅₀ thiol groups, or any combination thereof. Another subembodiment of this aspect of the disclosure is realized when the linking group comprises one or more —(OCH₂CH₂)ₚ— groups wherein p is an integer, for example, from about 1 to about 100, about 1 to about 80, about 1 to about 60, about 1 to about 40, about 1 to about 20, about 1 to about 10, about 1 to about 5, about 5 to about 100, about 5 to about 80, about 5 to about 60, about 5 to about 40, about 5 to about 20, about 5 to about 10, about 10 to about 100, about 10 to about 80, about 10 to about 60, about 10 to about 40, about 10 to about 20, about 20 to about 100, about 20 to about 80, about 20 to about 60, about 20 to about 40, about 40 to about 100, about 40 to about 80, about 40 to about 60, about 60 to about 100, about 60 to about 80, or about 80 to about 100 amino acid residues. In some embodiments, p is an integer from about 10 to about 40. In some embodiments, p is an integer from about 20 to about 40. In some embodiments, p is an integer from about 25 to about 35.

Another embodiment of the disclosure is realized when the compounds of Formula I, Formula I′ and Formula Ia, or a pharmaceutically acceptable salt thereof is linked to a chelating moiety containing one or more of the imaging agents described herein.

The compounds of the disclosure may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all the possible optical isomers and diastereomers in mixtures and as pure or partially purified compounds are included within the ambit of this disclosure. Unless a specific stereochemistry is indicated, the present disclosure is meant to encompass all such isomeric forms of these compounds.

The independent syntheses of these diastereomers or their chromatographic separations may be achieved as known in the art by appropriate modification of the methodology disclosed herein. Their absolute stereochemistry may be determined, amongst other methods, by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

In the compounds of Formula I, I′, or Ia, the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure may include all suitable isotopic variations of the compounds of generic Formulae I, I′, or Ia. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. For purposes of this disclosure when a compound is said to be “not deuterated” it means not enriched in deuterium beyond the background state. Isotopically-enriched compounds within generic Formulae I, I′, or Ia can be prepared without undue experimentation by conventional techniques well known to those skilled in the art or by processes analogous to those described in the Schemes and Examples herein using appropriate isotopically-enriched reagents and/or intermediates.

When a compound of the disclosure can form tautomers, all such tautomeric forms are also included within the scope of the present disclosure. For example, compounds including carbonyl —CH₂C(O)— groups (keto forms) may undergo tautomerism to form hydroxyl —CH═C(OH)— groups (enol forms). Both keto and enol forms, where present, are included within the scope of the present disclosure.

When any variable (e.g., R⁵, etc.) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is bicyclic, it is intended that the bond be attached to any of the suitable atoms on either ring of the bicyclic moiety.

It is understood that one or more silicon (Si) atoms can be incorporated into the compounds of the instant disclosure in place of one or more carbon atoms by one of ordinary skill in the art to provide compounds that are chemically stable and that can be readily synthesized by techniques known in the art from readily available starting materials. Carbon and silicon differ in their covalent radius leading to differences in bond distance and the steric arrangement when comparing analogous C-element and Si-element bonds. These differences lead to subtle changes in the size and shape of silicon-containing compounds when compared to carbon. One of ordinary skill in the art would understand that size and shape differences can lead to subtle or dramatic changes in potency, solubility, lack of off-target activity, packaging properties, and so on. (Diass, J. O. et al. Organometallics (2006) 5:1188-1198; Showell, G. A. et al. Bioorganic & Medicinal Chemistry Letters (2006) 16:2555-2558).

It is understood that substituents and substitution patterns on the compounds of the instant disclosure can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be understood as meaning that the group in question is either unsubstituted or may be substituted with one or more substituents.

Absolute stereochemistry is illustrated by the use of hashed and solid wedge bonds. As shown in Illus-I and Illus-II. Accordingly, the methyl group of Illus-I is emerging from the page of the paper and the ethyl group in Illus-II is descending into the page, where the cyclohexene ring resides within the plane of the paper. It is assumed that the hydrogen on the same carbon as the methyl group of Illus-I descends into the page and the hydrogen on the same carbon as the ethyl group of Illus-II emerges from the page. The convention is the same where both a hashed and solid rectangle are appended to the same carbon as in Illus-III, the methyl group is emerging from the plane of the paper and the ethyl group is descending into the plane of the paper with the cyclohexene ring in the plane of the paper.

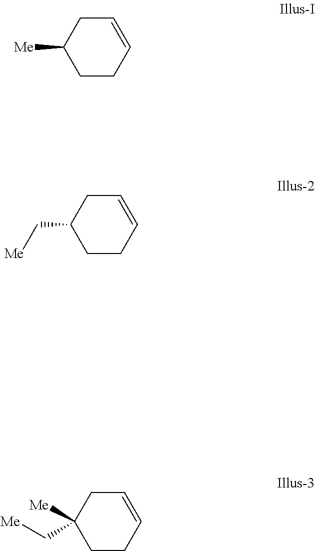 [SMILES: C[C@H]1CC=CCC1 ; CC[C@@H]1CC=CCC1 ; CC[C@]1(C)CC=CCC1]

As is conventional, unless otherwise noted in accompanying text, ordinary “stick” bonds or “wavy” bonds indicate that all possible stereochemistry is represented, including, pure compounds, mixtures of isomers, and racemic mixtures.

As used herein, unless otherwise specified, the following terms have the following meanings:

The phrase “at least one” used in reference to the number of components comprising a composition, for example, “at least one pharmaceutical excipient” means that one member of the specified group is present in the composition, and more than one may additionally be present. Components of a composition are typically aliquots of isolated pure material added to the composition, where the purity level of the isolated material added into the composition is the normally accepted purity level for a reagent of the type.

Whether used in reference to a substituent on a compound or a component of a pharmaceutical composition the phrase “one or more”, means the same as “at least one”;

“Effective amount” or “therapeutically effective amount” is meant to describe the provision of an amount of at least one active compound or pharmaceutical agent of the disclosure or of a composition comprising at least one compound or pharmaceutical agent of the disclosure that elicits the biological or medicinal response that is being sought in a tissue, system, animal, individual or human, or which is effective in treating or inhibiting a disease or condition described herein, and thus produce the desired therapeutic, ameliorative, inhibitory or preventative effect. For example, in treating central nervous system diseases or disorders with one or more of the compounds described herein “effective amount” (or “therapeutically effective amount”) means, for example, providing the amount of at least one compound of Formula I, Formula Ia, or a pharmaceutically acceptable salt thereof that results in a therapeutic response in a patient afflicted with a central nervous system disease or disorder (“condition”), including a response suitable to manage, alleviate, ameliorate, or treat the condition or alleviate, ameliorate, reduce, or eradicate one or more symptoms attributed to the condition and/or long-term stabilization of the condition, for example, as may be determined by the analysis of pharmacodynamic markers or clinical evaluation of patients afflicted with the condition;

“Patient” and “subject” means an animal, such as a mammal (e.g., a human being) and is preferably a human being;

“Prodrug” means compounds that are rapidly transformed, for example, by hydrolysis in blood, in vivo to the parent compound; a thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference; the scope of this disclosure includes prodrugs of the novel compounds of this disclosure;

The term “substituted” means that one or more of the enumerated substituents can occupy one or more of the bonding positions on the substrate typically occupied by “—H”, provided that such substitution does not exceed the normal valency rules for the atom in the bonding configuration presented in the substrate, and that the substitution ultimately provides a stable compound, which is to say that such substitution does not provide compounds with mutually reactive substituents located geminal or vicinal to each other; and wherein the substitution provides a compound sufficiently robust to survive isolation to a useful degree of purity from a reaction mixture.

Where optional substitution of a moiety is described (e.g., “optionally substituted”) the term means that if substituents are present, one or more of the enumerated substituents for the specified substrate can be present on the substrate in a bonding position normally occupied by the default substituent normally occupying that position. For example, a default substituent on the carbon atoms of an alkyl moiety is a hydrogen atom, an optional substituent can replace the default substituent.

As used herein, unless otherwise specified, the following terms used to describe moieties, whether comprising the entire definition of a variable portion of a structural representation of a compound of the disclosure or a substituent appended to a variable portion of a structural representation of a group of compounds of the disclosure have the following meanings, and unless otherwise specified, the definitions of each term (i.e., moiety or substituent) apply when that term is used individually or as a component of another term (e.g., the definition of aryl is the same for aryl and for the aryl portion of arylalkyl, alkylaryl, arylalkynyl moieties, and the like); moieties are equivalently described herein by structure, typographical representation or chemical terminology without intending any differentiation in meaning, for example, an “acyl” substituent may be equivalently described herein by the term “acyl”, by typographical representations “R′—(C═O)—” or “R′—C(O)—”, or by a structural representation:

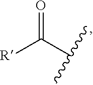

equally, with no differentiation implied using any or all of these representations;

The term “alkyl” (including the alkyl portions of other moieties, such as trifluoromethyl-alkyl- and alkoxy-) means a straight or branched aliphatic hydrocarbon moiety comprising up to about 20 carbon atoms (for example, a designation of “C₁₋₂₀-alkyl” indicates an aliphatic hydrocarbon moiety of from 1 to 20 carbon atoms). In some embodiments, alkyls preferably comprise up to about 10 carbon atoms, unless the term is modified by an indication that a shorter chain is contemplated, for example, an alkyl moiety of from 1 up to 8 carbon atoms is designated herein “C₁₋₈-alkyl”. Where the term “alkyl” is indicated with two hyphens (i.e., “-alkyl-” it indicates that the alkyl moiety is bonded in a manner that the alkyl moiety connects the substituents on either side of it, for example, “-alkyl-OH” indicates an alkyl moiety connecting a hydroxyl moiety to a substrate.

Unless otherwise noted, “Natural amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

The term “Non-natural amino acid” refers to a molecule which is structurally similar to an amino acid and which can be substituted for an amino acid in the formation of a macrocycle. Non-natural analogs include, without limitation, compounds which are structurally identical to an amino acid, as defined herein, except for the inclusion of one or more additional methylene groups between the amino and carboxyl group (e.g., α-amino, β-carboxy acids), or for the substitution of the amino or carboxy group by a similarly reactive group (e.g., substitution of the primary amine with a secondary or tertiary amine, or substitution or the carboxy group with an ester).

Substitutions on certain amino acids can be conserved, i.e., those where substitutions of amino acids with other amino acids have similar characteristics (e.g., charge, side-chain size, hydrophobicity/hydrophilicity, backbone conformation and rigidity, etc.), such that the changes can frequently be made without drastically altering the biological activity of the protein. Those of skill in this art recognize that, in general, single amino acid substitutions in non-essential regions of a polypeptide do not substantially alter biological activity (see, e.g., Watson et al. Molecular Biology of the Gene, The Benjamin/Cummings Pub. Co., p. 224 (4th Ed.) (1987)). In addition, substitutions of structurally or functionally similar amino acids are less likely to disrupt biological activity. Exemplary conservative substitutions are set forth in Table Y.

TABLE Y

| Original | Conservative | Original | Conservative |
|---|---|---|---|
| residue | substitution | residue | substitution |
| Ala (A) | Gly; Ser | Leu (L) | Ile; Val |
| Arg (R) | Lys; His | Lys (K) | Arg; His |
| Asn (N) | Gln; His | Met (M) | Leu; Ile; Tyr |
| Asp (D) | Glu; Asn | Phe (F) | Tyr; Met; Leu |
| Cys (C) | Ser; Ala | Pro (P) | Ala |
| Gln (Q) | Asn | Ser (S) | Thr |
| Glu (E) | Asp; Gln | Thr (T) | Ser |
| Gly (G) | Ala | Trp (W) | Tyr; Phe |
| His (H) | Asn; Gln | Tyr (Y) | Trp; Phe |
| Ile (I) | Leu; Val | Val (V) | Ile; Leu |

“Dose”, “dosage”, “unit dose”, “unit dosage”, “effective dose” and related terms refer to physically discrete units that contain a predetermined quantity of active ingredient (e.g., peptidomimetic macrocycle) calculated to produce a desired therapeutic effect (e.g., death of cancer cells). These terms are synonymous with the therapeutically effective amounts and amounts sufficient to achieve the stated goals of the methods disclosed herein.

The term “cycloalkyl” means a moiety having a main hydrocarbon chain forming a mono- or bicyclo-cyclic aliphatic moiety comprising at least 3 carbon atoms (the minimum number necessary to provide a monocyclic moiety) up to the maximum number of specified carbon atoms, generally 8 for a monocyclic moiety and 10 for a bicyclic moiety, inclusive of spirocyclic moieties. Examples of cycloalkyl moieties include, but are not limited to, cyclopropyl, cyclobutyl, cyclopentyl or cyclohexyl. The term “cycloalkyl” also includes non-aromatic, fused multicyclic ring system comprising up to 20 carbon atoms which may optionally be substituted as defined herein for “alkyl” generally. Suitable multicyclic cycloalkyls are, for example, but are not limited to: 1-decalin; norbornyl; adamantly; and the like;

As used herein, the term “alkylene” refers to a saturated linear or branched aliphatic hydrocarbon group having two residues derived from the removal of two hydrogen atoms from the same carbon atom or two different carbon atoms of the parent alkane. The alkylene is a linear or branched group having 1 to 20 carbon atoms, preferably 1 to 12 carbon atoms, and more preferably 1 to 6 carbon atoms. Non-limiting examples are methylene, ethylene, propylene, butylene, pentylene, and the like.

As used herein, when the term “alkyl” is modified by “substituted” or “optionally substituted”, it means that one or more C—H bonds in the alkyl moiety group is substituted, or optionally may be substituted, by a substituent bonded to the alkyl substrate which is called out in defining the moiety.

The term “cycloalkyl” refers to a saturated or partially unsaturated monocyclic or polycyclic hydrocarbon substituent group having 3 to 20 carbon atoms, preferably 3 to 12 carbon atoms, more preferably 3 to 10 carbon atoms, and most preferably 3 to 8 carbon atoms. Non-limiting examples of monocyclic cycloalkyl include cyclopropyl, cyclobutyl, cyclopentyl, cyclopentenyl, cyclohexyl, cyclo-hexenyl, cyclohexadienyl, cycloheptyl, cycloheptatrienyl, cyclooctyl and the like. Polycyclic cycloalkyl includes a cycloalkyl having a spiro ring, fused ring or bridged ring.

Where a structural formula represents bonding between a moiety and a substrate using a bonding line that terminates in the middle of the structure, for example the following representations:

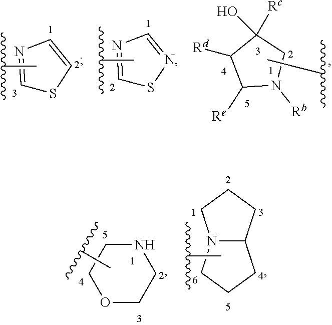

whether or not numbered the structure indicates that unless otherwise defined the moiety may be bonded to the substrate through any of available ring atom, for example, the numbered atoms of the example moieties.

The term “aryl” refers to a 6 to 14 membered all-carbon monocyclic ring or polycyclic fused ring (i.e., each ring in the system shares an adjacent pair of carbon atoms with another ring in the system) having a conjugated electron system, preferably a 6 to 10 membered aryl, for example, phenyl and naphthyl, and preferably phenyl.

The term “heteroaryl” refers to an aromatic 5-8 membered monocyclic, 8-12 membered bicyclic, or 11-14 membered tricyclic ring system having 1-3 heteroatoms for monocyclic. 1-6 heteroatoms for bicyclic, or 1-9 heteroatoms for tricyclic, said heteroatoms selected from O, N, or S (e.g., carbon atoms and 1-3, 1-6, or 1-9 heteroatoms of N, O, or S for monocyclic, bicyclic, or tricyclic, respectively). Non-limiting examples of heteroaryls are imidazolyl, pyridyl, pyrazolyl, pyrimidinyl, furanyl, oxazolyl, triazolyl, oxadiazolyl, and thiophenyl. The heteroaryl groups herein described may also contain fused rings that share a common carbon-carbon bond, such as indolyl.

The term “heterocyclyl” (or heterocycloalkyl) means anon-aromatic saturated monocyclic or multicyclic ring system comprising 3 to 10 ring atoms, preferably 5 to 10 ring atoms, in which one or more of the atoms in the ring system is an element other than carbon, for example nitrogen (e.g., azetidinyl, piperidyl, pyrrolidinyl, tetrahydroisoquinolinyl,), oxygen (e.g., furanyl and tetrahydropyranyl) or sulfur (e.g., tetrahydrothiopheneyl and tetrahydrothiopyranyl); and wherein the heteroatoms can be alone or in combination provided that the moiety does not contain adjacent oxygen and/or sulfur atoms present in the ring system. The heterocyclyl can be optionally substituted by one or more independently selected substituents.

The nitrogen or sulfur atom of the heterocyclyl can be optionally oxidized to the corresponding N-oxide, S-oxide or S,S-dioxide (SO2). Non-limiting examples of suitable monocyclic heterocyclyl rings include azetidinyl, piperidyl, pyrrolidinyl, piperazinyl, morpholinyl

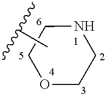

(where unless otherwise noted the moiety is bonded to the substrate through any of ring carbon atoms C2, C3, C5, or C6), thiomorpholinyl, thiazolidinyl, 1,3-dioxolanyl, 1,4-dioxanyl, tetrahydrofuranyl, tetrahydrothiophenyl, tetrahydrothiopyranyl, and the like; and bicyclic rings such as tetrahydroisoquinolinyl

The term “solvate” refers to a pharmaceutically acceptable solvate formed by a compound of the present disclosure with one or more solvent molecule(s). Non-limiting examples of solvent molecules include water, ethanol, acetonitrile, isopropanol, DMSO, ethyl acetate.

The term “halogen” means fluorine, chlorine, bromine, or iodine; preferred halogens, unless specified otherwise where the term is used, are fluorine, chlorine and bromine, a substituent which is a halogen atom means —F, —Cl, —Br, or —I, and “halo” means fluoro, chloro, bromo, or iodo substituents bonded to the moiety defined, for example, “haloalkyl” means an alkyl, as defined above, wherein one or more of the bonding positions on the alkyl moiety typically occupied by hydrogen atoms are instead occupied by a halo group, perhaloalkyl (or “fully halogenated” alkyl) means that all bonding positions not participating in bonding the alkyl substituent to a substrate are occupied by a halogen, for example, where the alkyl is selected to be methyl, the term perfluoroalkyl means —CF₃;

The term “hydroxyl” and “hydroxy” means an HO— group, “hydroxyalkyl” means a substituent of the formula: “HO-alkyl-”, wherein the alkyl group is bonded to the substrate and may be substituted or unsubstituted as defined above: preferred hydroxyalkyl moieties comprise a lower alkyl: Non-limiting examples of suitable hydroxyalkyl groups include hydroxymethyl and 2-hydroxyethyl; and

The bonding sequence is indicated by hyphens where moieties are represented in text, for example -alkyl, indicates a single bond between a substrate and an alkyl moiety, -alkyl-X, indicates that an alkyl group bonds an “X” substituent to a substrate, and in structural representation, bonding sequence is indicated by a wavy line terminating a bond representation, for example:

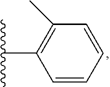 [SMILES: *c1ccccc1C]

indicates that the methylphenyl moiety is bonded to a substrate through a carbon atom ortho to the methyl substituent, while a bond representation terminated with a wavy line and drawn into a structure without any particular indication of an atom to which it is bonded indicates that the moiety may be bonded to a substrate via any of the atoms in the moiety which are available for bonding as described in the examples above.

The line -, as a bond generally indicates a mixture of, or either of, the possible isomers, e.g., containing (R)- and (S)-stereochemical configuration.

Furthermore, unwedged-bolded or unwedged-hashed lines are used in structures containing multiple stereocenters in order to depict relative configuration where it is known. For example:

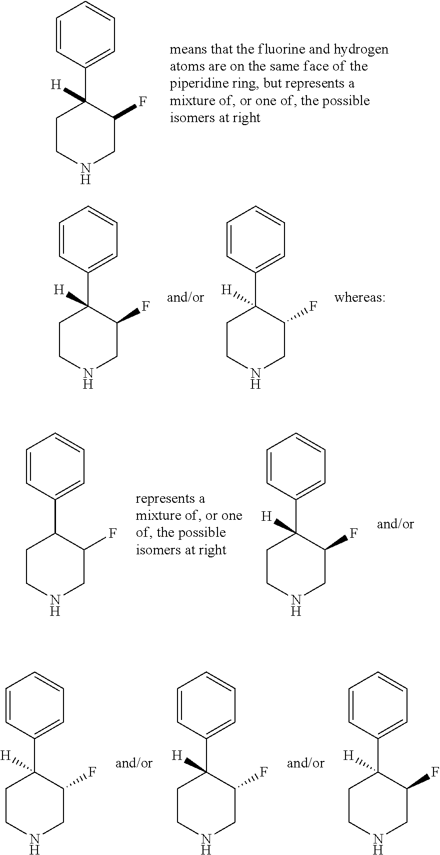

In all cases, compound name(s) accompany the structure drawn and are intended to capture each of the stereochemical permutations that are possible for a given structural isomer based on the synthetic operations employed in its preparation. Lists of discrete stereoisomers that are conjoined using or indicate that the presented compound (e.g., ‘Example number’) was isolated as a single stereoisomer, and that the identity of that stereoisomer corresponds to one of the possible configurations listed. Lists of discrete stereoisomers that are conjoined using and indicate that the presented compound was isolated as a racemic mixture or diastereomeric mixture.

A specific absolute configuration is indicated by use of a wedged-bolded or wedged-hashed line. Unless a specific absolute configuration is indicated, the present disclosure is meant to encompass all such stereoisomeric forms of these compounds.

In this specification, where there are multiple oxygen and/or sulfur atoms in a ring system, there cannot be any adjacent oxygen and/or sulfur present in said ring system.

As well known in the art, a bond drawn from a particular atom wherein no moiety is depicted at the terminal end of the bond indicates a methyl group bound through that bond to the atom, unless stated otherwise. For example:

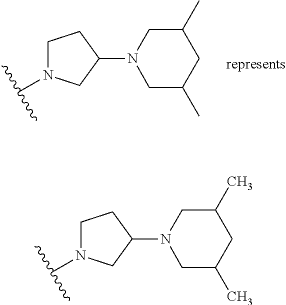 [SMILES: *N1CCC(N2CC(C)CC(C)C2)C1 ; *N1CCC(N2CC(C)CC(C)C2)C1]

Unsatisfied valences in the text, schemes, examples, structural formulae, and any Tables herein are assumed to have a hydrogen atom or atoms of sufficient number to satisfy the valences.

One or more compounds of the disclosure may also exist as, or optionally be converted to, a solvate. Preparation of solvates is generally known. Thus, for example, M. Caira et al., J Pharmaceutical Sci., 93(3), 601-611 (2004) describe the preparation of the solvates of the antifungal fluconazole in ethyl acetate as well as from water. Similar preparations of solvates, and hemisolvate, including hydrates (where the solvent is water or aqueous-based) and the like are described by E. C. van Tonder et al., AAPS PharmSciTech., 5(1), article 12 (2004); and A. L. Bingham et al., Chem. Commun., 603-604 (2001). A typical, non-limiting, process involves dissolving the inventive compound in desired amounts of the desired solvent (for example, an organic solvent, an aqueous solvent, water or mixtures of two or more thereof) at a higher than ambient temperature, and cooling the solution, with or without an antisolvent present, at a rate sufficient to form crystals which are then isolated by standard methods. Analytical techniques such as, for example I.R. spectroscopy, show the presence of the solvent (including water) in the crystals as a solvate (or hydrate in the case where water is incorporated into the crystalline form).

This disclosure also includes the compounds of this disclosure in isolated and purified form obtained by routine techniques. Polymorphic forms of the compounds of Formula I and Formula La and of the salts, solvates and prodrugs of the compounds of Formula I and Formula Ia are intended to be included in the present disclosure. Certain compounds of the disclosure may exist in different isomeric forms (e.g., enantiomers, diastereoisomers, atropisomers). The inventive compounds include all isomeric forms thereof, both in pure form and admixtures of two or more, including racemic mixtures.

In the same manner, unless indicated otherwise, presenting a structural representation of any tautomeric form of a compound which exhibits tautomerism is meant to include all such tautomeric forms of the compound. Accordingly, where compounds of the disclosure, their salts, and solvates and prodrugs thereof, may exist in different tautomeric forms or in equilibrium among such forms, all such forms of the compound are embraced by, and included within the scope of the disclosure. Examples of such tautomers include, but are not limited to, ketone/enol tautomeric forms, imine-enamine tautomeric forms, and for example heteroaromatic forms such as the following moieties:

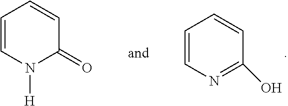 [SMILES: O=c1cccc[nH]1 ; Oc1ccccn1]

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives wherein the parent compound is modified by making acid or base salts thereof. Salts in the solid form may exist in more than one crystal structure and may also be in the form of hydrates. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as formic, hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Salts derived from inorganic bases include aluminum, ammonium, calcium, copper, ferric, ferrous, lithium, magnesium, manganic salts, manganous, potassium, sodium, zinc, and the like.

When the compound of the present disclosure is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. In one aspect of the disclosure the salts are citric, hydrobromic, hydrochloric, maleic, phosphoric, sulfuric, fumaric, and tartaric acids. Similarly, the salts of the acidic compounds are formed by reactions with the appropriate inorganic or organic base.

The terms “treating” or “treatment” (of, e.g., a disease, disorder, or conditions or associated symptoms, which together or individually may be referred to as “indications”) as used herein include: inhibiting the disease, disorder or condition, i.e., arresting or reducing the development of the disease or its biological processes or progression or clinical symptoms thereof; or relieving the disease, i.e., causing regression of the disease or its biological processes or progression and/or clinical symptoms thereof. “Treatment” as used herein also refers to control, amelioration, or reduction of risks to the subject afflicted with a disease, disorder or condition in which a tumor is involved. The terms “preventing” or “prevention” or “prophylaxis” of a disease, disorder or condition as used herein includes: impeding the development or progression of clinical symptoms of the disease, disorder, or condition in a mammal that may be exposed to or predisposed to the disease, disorder or condition but does not yet experience or display symptoms of the disease, and the like.

As would be evident to those skilled in the art, subjects treated by the methods described herein are generally mammals, including humans and non-human animals (e.g., laboratory animals and companion animals). The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising a compound of the disclosure or a pharmaceutically acceptable salt thereof, together with one or more additional specified ingredients in the specified amounts, as well as any product which results, directly or indirectly, from combination of the specified ingredients in the specified amounts. Such term in relation to a pharmaceutical composition, is intended to encompass a product comprising the active ingredient(s), which include a compound of the disclosure or a pharmaceutically acceptable salt thereof, optionally together with one or more additional active ingredients, and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure, or a pharmaceutically acceptable salt thereof, and a pharmaceutically acceptable carrier. By “pharmaceutically acceptable” it is meant the carrier, diluent or excipient must be compatible with the other ingredients of the formulation and not deleterious to the recipient thereof.

In some embodiments, the compound of Formula I, Formula I′, or Formula La is a polypeptide that binds granzyme B, wherein the polypeptide comprises an amino acid sequence having at least 90% sequence identity to the sequence of Compound 1 in Table 1. In some embodiments, the compound of Formula I, Formula I′, or Formula La is a polypeptide that binds granzyme B, wherein the polypeptide comprises an amino acid sequence having at least 95% sequence identity to the sequence of Compound 1 in Table 1. In some embodiments, the compound of Formula I, Formula I′, or Formula Ia is a polypeptide that binds granzyme B, wherein the polypeptide comprises an amino acid sequence having at least 98% sequence identity to the sequence of Compound 1 in Table 1. In some embodiments, the compound of Formula I, Formula I′, or Formula Ia is a polypeptide that binds granzyme B, wherein the polypeptide comprises an amino acid sequence having at least 99% sequence identity to the sequence of Compound 1 in Table 1. In some embodiments, the compound of Formula I, Formula I′, or Formula La is a polypeptide that binds granzyme B, wherein the polypeptide comprises an amino acid sequence having at least 99.5% sequence identity to the sequence of Compound 1 in Table 1.

As noted above, additional embodiments of the present disclosure are each directed to a method for the treatment a disease, disorder, or condition, or one or more symptoms thereof (“indications”) which method comprises administering to a subject in need of such treatment a therapeutically effective amount of a compound of the disclosure, or a pharmaceutically acceptable salt thereof or a pharmaceutical composition comprising said compound or salt thereof. Thus, an embodiment of this disclosure is a method of treating a disease, disorder, or condition, or one or more symptoms thereof (“indications”) where granzyme B is implicated.

Another embodiment of this aspect of the disclosure is realized when the disease is selected from an autoimmune disorder, inflammatory disorder, skin disorder, cancer and cardiovascular disorder. A subembodiment of this aspect of the disclosure relates to a disease that is cancer selected from breast cancer, ovarian cancer, cervical cancer, uterine cancer, prostate cancer, kidney cancer, urethral cancer, bladder cancer, liver cancer, stomach cancer, endometrial cancer, salivary gland cancer, esophageal cancer, melanoma, glioma, neuroblastoma, sarcoma, lung cancer (for example, small cell lung cancer and non-small cell lung cancer) colon cancer, rectal cancer, colorectal cancer, leukemia (for example, acute lymphocytic leukemia, acute myeloid leukemia, acute promyelocytic leukemia, chronic myeloid leukemia, chronic lymphocytic leukemia), bone cancer, skin cancer, thyroid cancer, pancreatic cancer, and lymphoma (for example. Hodgkin's lymphoma, non-Hodgkin's lymphoma, or recurrent anaplastic large cell lymphoma). Another subembodiment of this aspect of the disclosure relates to a method of treating or preventing cancer, in a subject in need thereof, said method comprising administering to a subject in need of such treatment a therapeutically effective amount of a compound of Formula I, Formula I′. Formula Ia, or a pharmaceutically acceptable salt or solvate thereof, or a pharmaceutical composition comprising said compound, salt or solvate thereof. In one such embodiment, the subject is a human.

Another embodiment of the disclosure relates to methods of imaging granzyme B. A subembodiment of this aspect of the disclosure relates to a method wherein imaging is performed in a cell, a tissue, a cell sample, a tissue sample, or a subject. As used herein, the term “subject,” refers to any animal, including mammals (e.g., humans, domestic animals, farm animals, etc.) and invertebrates (e.g., fish). Another embodiment of the disclosure relates to a method of imaging granzyme B in a cell or tissue, comprising contacting the cell or tissue with a compound of Formula I. Formula I′, Formula Ia, or a pharmaceutically acceptable salt thereof and imaging the cell or tissue with a suitable imaging technique to image the granzyme B in the cell or tissue. A subembodiment of this aspect of the disclosure is realized when the compound of Formula I, Formula 1′. Formula Ia, or a pharmaceutically acceptable salt thereof comprises an imaging agent.

Another embodiment of the disclosure relates to a method of imaging an immune response in a subject, comprising administering to the subject a compound of Formula I, Formula I′, Formula Ia, or a pharmaceutically acceptable salt thereof, and imaging the subject with a suitable imaging technique to image the granzyme B in the cell or tissue. A subembodiment of this aspect of the disclosure is realized when the compound of Formula I, Formula I′, Formula Ia, or a pharmaceutically acceptable salt thereof comprises an imaging agent.

Another embodiment of the disclosure relates to a method of monitoring an immune response in the treatment of a disease in a subject, comprising administering to the subject a compound of Formula I, Formula I′, Formula La, or a pharmaceutically acceptable salt thereof, and imaging the subject with a suitable imaging technique to image the granzyme B in the cell or tissue. A subembodiment of this aspect of the disclosure is realized when the compound of Formula I, Formula I′. Formula La, or a pharmaceutically acceptable salt thereof comprises an imaging agent.

Another embodiment of the disclosure is realized when the compounds are used as imaging agents in diseases selected from graft-versus-host disease, rheumatoid arthritis, systemic lupus erythematosus. Hashimoto's thyroiditis, multiple sclerosis, myasthenia gravis, type I diabetes, uveitis, posterior uveitis, allergic encephalomyelitis, glomerulonephritis, rheumatic fever, post-infectious glomerulonephritis, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia areata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, coeliac diseases, proctitis, eosinophilic gastroenteritis, mastocytosis, Crohn's disease, ulcerative colitis, migraine, rhinitis, eczema, interstitial nephritis, Goodpasture's syndrome, hemolyticuremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, der-matomyositis, leukoderma vulgaris, ichthyosis vulgaris, photoallergic sensitivity, cutaneous T cell lymphoma, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia, alopecia senilis by preventing epilation, alopecia senilis by providing hair germination and/or promoting hair generation and hair growth, muscular dystrophy, pyoderma, Sezary's syndrome, Addison's disease, ischemia-reperfusion injury of organs, transplantation disease, ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis, sepsis, pancreatitis, aging, carcinogenesis, metastasis of carcinoma and hypobaropathy, histamine or leukotriene-C4 release associated diseases, Behcet's disease, autoimmune hepatitis, primary biliary cirrhosis, sclerosing cholangitis, partial liver resection, acute liver necrosis, necrosis caused by toxin, viral hepatitis, shock, anoxia, B-virus hepatitis, non-A/non-B hepatitis, cirrhosis, alcoholic cirrhosis, hepatic failure, fulminant hepatic failure, late-onset hepatic failure, acute-on-chronic liver failure, cyto-megalovirus infection, HCMV infection, AIDS, senile dementia, trauma, chronic bacterial infection, malignancy of lymphoid origin, acute lymphocytic leukemia, chronic lymphocytic leukemia, acute lymphocytic lymphoma, and chronic lymphocytic lymphoma.

Another embodiment of the disclosure is realized when the disease is selected from systemic lupus erythematosis, chronic rheumatoid arthritis, type I diabetes mellitus, inflammatory bowel disease, biliary cirrhosis, uveitis, multiple sclerosis, Crohn's disease, ulcerative colitis, bullous pemphigoid, sarcoidosis, psoriasis, autoimmune myositis, Wegener's granulomatosis, ichthyosis, Graves ophthalmopathy, asthma, schleroderma and Sjogren's syndrome. Another embodiment of the disclosure is realized when the disease is selected from bone marrow rejection, organ transplant rejection, and graft-versus-host disease.

Another aspect of the disclosure relates to uses and processes for making such compounds, which may be suitable for imaging granzyme B.

The present disclosure includes within its scope prodrugs of the compounds of this disclosure. In general, such prodrugs will be functional derivatives of the compounds of this disclosure which are readily convertible in vivo into the required compound. Thus, in the methods of treatment of the present disclosure, the terms “administration of” or “administering a” compound shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this disclosure into the biological milieu.

The compounds described herein, or pharmaceutically acceptable salts and/or solvates thereof, may be administered singly, in combination with other compounds of the disclosure, and/or in cocktails combined with other therapeutic agents. The choice of therapeutic agents that can be co-administered with the compounds of the disclosure will depend, in part, on the condition being treated.

The compounds of the present disclosure may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, buccal or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals the compounds of the disclosure are effective for use in humans.

The pharmaceutical compositions for the administration of the compounds of this disclosure may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, solutions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated, or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and U.S. Pat. No. 4,265,874 to form osmotic therapeutic tablets for control release. Oral tablets may also be formulated for immediate release, such as fast melt tablets or wafers, rapid dissolve tablets or fast dissolve films.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or acetyl alcohol. Sweetening agents such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present disclosure may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions and the like, containing the compounds of the present disclosure are employed. Similarly, transdermal patches may also be used for topical administration.

The pharmaceutical composition and method of the present disclosure may further comprise other therapeutically active compounds as noted herein which are usually applied in the treatment of the above-mentioned pathological conditions.

In the treatment, prevention, control, amelioration, or reduction of risk of the conditions disclosed herein the phrase “therapeutically effective amount” refers to the amount of active compound or pharmaceutical agent that elicits the biological or medicinal response that is being sought in a tissue, system, animal, individual or human. In some embodiments, the dosage of the compound, or a pharmaceutically acceptable salt thereof, administered to a subject or individual is about 1 μg to about 2 g. about 1 μg to about 1000 mg, about 1 μg to about 500 mg, about 1 μg to about 100 mg, about 1 μg to about 50 mg, about 1 μg to about 1 mg, about 1 μg to about 500 μg, about 1 μg to about 100 μg, about 1 μg to about 10 μg, about 10 μg to about 2 g and the like. The compounds may be administered on a regimen of 1 to 4 times per day or may be administered once or twice per day.

Another embodiment of the disclosure is realized wherein the compounds of Formula I, Formula La, or a pharmaceutically acceptable salt thereof can be administered in combination with one or more of the additional therapeutic agents provided herein. A subembodiment of this aspect of the disclosure is realized when the additional therapeutic agents include, but are not limited to, anti-inflammatory agents, steroids, immuno-therapy agents, chemotherapeutic agents, and therapeutic antibodies.

Another embodiment of the disclosure is realized when administration of the therapeutic agent induces an immune response cell or tissue sample or subject. A subembodiment of this aspect of the disclosure is realized when the therapeutic agent is a compound of Formula I, Formula I′. Formula Ia, or a pharmaceutically acceptable salt thereof. A subembodiment of this aspect of the disclosure is realized when the therapeutic agent is a compound of Formula I, Formula I′. Formula Ia, or a pharmaceutically acceptable salt thereof, comprising a radioisotope (e.g., a therapeutic radioisotope). Another subembodiment of this aspect of the disclosure is realized when the therapeutic agent is a compound of Formula I, Formula I′, Formula Ia, or a pharmaceutically acceptable salt, comprising a toxic radioisotope. Another subembodiment of this aspect of the disclosure is realized when the toxic radioisotope is selected from alpha emitters (²¹¹At, ²¹²Pb, ²¹²Bi, ²¹³Bi, ²²⁵Ac, ²²⁷Th) and beta emitters (e.g., ⁹⁰Y ¹³¹I and ¹⁷⁷Lu, ¹⁶¹Tb).

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Methods for preparing the compounds of this disclosure are illustrated in the following Schemes and Examples. Starting materials are made according to procedures known in the art or as illustrated herein.

It will be appreciated by one skilled in the art that the processes described are not the exclusive means by which compounds provided herein may be synthesized and that a broad repertoire of synthetic organic reactions is available to be potentially employed in synthesizing compounds provided herein. The compounds of the present disclosure can be prepared according to the following illustrative schemes and specific examples, or modifications thereof, using readily available starting materials, reagents and conventional synthesis procedures. It is also possible to make use of variants which are themselves known to those of ordinary skill in this art but are not mentioned in detail. The general procedures for making the compounds claimed in this disclosure can be readily understood by one skilled in the art from viewing the following schemes and descriptions. Abbreviations used in the Experimentals may include, but are not limited to the following.

| | |
|---|---|
| aq | Aqueous |
| BOC | tert-butyloxycarbonyl |
| CV | Column Volume |
| DCM | Dichloromethane |
| DIPEA | N,N-Diisopropylethylamine |
| DMF | Dimethylformamide |
| eq. | Equivalent(s) |
| DMSO | Dimethyl sulfoxide |
| DODT | 3,6-dioxa-1,8-octanedithiol |
| Fmoc | Fluorenylmethyloxycarbonyl |
| GBq | gigaBecquerel |
| hr(s) | Hour(s) |
| HBTU | (2-(1H-benzotriazol-1-yl)-1,1,3,3- |
| | tetramethyluronium hexafluorophosphate |
| HOBt | Hydroxy benzotriazole |
| ¹H-NMR | Proton nuclear magnetic resonance |
| HPLC | High performance liquid chromatography |
| IPA | Isopropyl alcohol |
| LCMS | Liquid chromatography-mass spectrometry |
| min | Minute(s) |
| MeCN | Acetonitrile |
| MeOH | Methanol |
| MS | Mass spectrometry |
| m/z | Mass to charge ratio |
| Oxyma | Ethyl cyanohydroxyiminoacetate |
| RT | Room temperature |
| NMP | N-methylpyrrolidone |
| SPPS | Solid phase peptide synthesis |
| TFA | Trifluoroacetic acid |
| THF | Tetrahydrofuran |
| TFA | Trifluoroacetic acid |
| Rink amide MBHA | 4-(2′,4′-Dimethoxyphenyl-Fmoc-aminomethyl)- |
| | phenoxyacetamido-norleucyl-MBHA resin |
| Arg (R) | Arginine |
| Cys (C) | Cysteine |
| Asp (D) | Aspartic Acid |
| Gln (Q) | Glutamine |
| Glu (E) | Glutamic Acid |
| Gly (G) | Glycine |
| Lys (K) | Lysine |
| His (H) | Histidine |
| Leu (L) | Leucine |
| Ile (I) | Isoluecine |
| Asn (N) | Asparagine |
| Phe (F) | Phenylalanine |
| Pro (P) | Proline |
| Ser (S) | Serine |
| Thr (T) | Threonine |
| Trp (W) | Tryptophan |
| Tyr (Y) | Tyrosine |
| Val (V) | Valine |
| Ala (A) | Alanine |
| Met (M) | Methionine |

Table A lists non-natural amino acids with their abbreviation and structure.

TABLE A

| Amino acid | |
|---|---|
| abbreviation | Structure |
| CIAc | 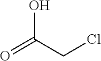 |
| NMeF | 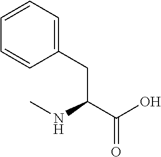 |
| NMeA | 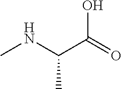 |

### General Peptide Synthesis Procedures

All reagents and solvents were purchased from commercial sources and used “as is” unless otherwise noted. Reaction progress and synthetic intermediate analysis were assessed by LCMS (UV detection with ESI, APCI, or other mass detection) when applicable using a MeCN/water gradient with either TFA, formic acid, or NH₄HCO₃ modifier. Silica gel and reverse-phase flash column chromatography were conducted with commercially available pre-packed columns. Reverse-phase preparative HPLC purification was performed on preparative HPLC instruments with UV and MS detection using a MeCN/water gradient with either TFA, formic acid, or NH₄OH modifier. Unless otherwise noted, all K_{D} data presented in tables refers to the surface plasmon resonance assay that is described in the Biological Assay section.

Peptides were synthesized using standard solid phase synthesis using Fmoc/tert-Bu chemistry as exemplified in Chan, W. C.; White, P. D. “Fmoc Solid-Phase Synthesis: a Practical Approach”, Oxford University Press, Oxford. 2000: Steward. J.; Young, J. “Solid Phase Peptide Synthesis”, Pierce Chemical Company, Rockford, 1984.; N. L. Benoiton, “Chemistry of Peptide Synthesis”, CRC Press, New York, 2006; and Lloyd-Williams, P.; Albericio, F. “Chemical Approaches to the Synthesis of Peptides and Proteins”, CRC Press, New York, 1997.

Peptide synthesis was completed using Protocol A or B outlined below unless otherwise indicated. The following protected natural amino acids were used: Fmoc-Ala-OH; Fmoc-Arg(Pbf)-OH; Fmoc-Asn(Trt)-OH; Fmoc-Cys(Trt)-OH; Fmoc-Gln(Trt)-OH; Fmoc-Gly-OH; Fmoc-Glu(OtBu)-OH; Fmoc-His(Trt)-OH; Fmoc-Ile-OH; Fmoc-Leu-OH; Fmoc-Lys(Boc)-OH; Fmoc-Phe-OH; Fmoc-Pro-OH; Fmoc-Ser(t-Bu)-OH; Fmoc-Thr(t-Bu)-OH; Fmoc-Trp-OH; Fmoc-Tyr(t-Bu)-OH; and Fmoc-Val-OH. All non-natural amino acids in the sequence contained an Fmoc-α-N protected amine, and relevant side-chains were protected with Boc- or tBu-protecting groups.

### Protocol A:

Step 1—synthesis of linear peptide: N^{α}-Fmoc protected Rink amide MBHA resin (0.7 mmol, 1.0 g) was swollen in DCM (15 mL) for 1.0 hours in a glass sintered vessel equipped with a frit. The mixture was filtered and 20% piperidine in DMF (15 mL) was added into the resin. The mixture was shaken on an orbital shaker at room temperature for 5 mins. The solution was drained and the resin was treated again with 20% piperidine/DMF solution for 20 minutes on an orbital shaker. The mixture was filtered and the peptidyl resin was washed subsequently with DMF (5×15 mL), then DCM (5×15 mL), and then DMF(5×15 mL). A Kaiser test is performed on the beads to confirm the presence of free amine. The resin was treated with a solution containing:

Fmoc-protected amino acid (3.5 mmol), DMF (10 mL), N-methylmorpholine (7 mmol), and HBTU (3.5 mmol) or

Fmoc-protected amino acid (3.5 mmol), DMF (10 mL), HBTU (7 mmol), and HOBt (3.5 mmol), and N,N-diisopropylethylamine (7 mmol) or

Fmoc-protected amino acid (3.5 mmol), DMF (10 mL), N,N′-diisopropylcarbodiimide (7 mmol), and HOBt (3.5 mmol).

The resulting suspension was kept at room temperature for 2 h on an orbital shaker. After the Kaiser test indicated the completion of the coupling reaction. The mixture was filtered, and the peptidyl resin was washed with DMF (5×15 mL), DCM (5×15 mL), DMF(5×15 mL). Subsequent Fmoc-deprotection and amino acid coupling steps were repeated as described above. N-terminal chloroacetylation was performed with 10% chloroacetic anhydride/DCM in the presence of 6 equivalents (with respect to resin substitution) of N,N-diisopropylethylamine. Following synthesis, the resin was washed with DMF and DCM (3×15 mL), followed by methanol (20 mL) and diethyl ether (2×20 mL). Finally, the resin was dried under vacuum overnight.

Step 2—Cleavage and Deprotection: Cleavage of the peptide from the solid support was achieved by treating the peptide-resin (2.5 g) with reagent cocktail (95% TFA/2.5% TIS (triisopropylsilane)/2.5% Water/2.5% DODT; 10 mL/g peptidyl-resin) at room temperature for 3 h. The cleavage mixture was collected by filtration and the resin was washed with TFA. For precipitation of the peptide, 5 mL of cleavage mixture containing peptide was precipitated in 45 mL of cold (0° C.) anhydrous ether. The precipitated peptide was centrifuged (3800 rpm), and the supernatant ether was removed. Fresh ether was added to the peptide and re-centrifuged. This process was repeated three times. The precipitated peptide was then lyophilized under high vacuum overnight to give the linear crude peptide solid.

Step 3—Peptide Cyclization: Linear crude peptide was dissolved in DMSO to a concentration of 5 mM and the pH was adjusted to 8-9 (monitoring with water-wet pH paper) by adding triethylamine or N,N′-diisopropylethylamine. The reaction was agitated at room temperature for 16 hours, then neutralized by addition of TFA to pH~7. The reaction solution was then taken directly to purification.

Step 4—Peptide Purification: The cyclized crude material was purified using preparative HPLC using a Phenomenex Luna C18 5u 100A 250*21.2 mm column and linear gradients of acetonitrile in water, both buffered with 0.1% TFA. The HPLC fractions containing a pure peptide product were pooled and lyophilized.

### Protocol B:

Step 1—Synthesis of linear peptide: The peptide sequence was assembled by solid phase synthesis on a Microwave Liberty Blue synthesizer (CEM, Matthews, NC). The synthesis was started using 100 pmol of Novabiochem Rink Amide AM resin LL, 100-200 mesh, 0.29 mmol/g (CEM). Each amino acid was coupled in 9-fold excess as a 0.2 M solution in DMF, which was activated using a 9-fold excess of 0.5 M diisopropylcarbodiimide (DIC) and 1 M Oxyma in DMF. Fmoc-deprotection steps were performed with 20% pyrrolidine in DMF with 0.1M HOBT for 60 sec at 90° C. Single and double couplings were performed at 90° C. with 5 min coupling times, except Fmoc-His (Trt)-OH, which was coupled at 50° C. Acylation of secondary amino acids in the sequence was performed by double coupling. Aspartic acid was coupled as the Fmoc-Asp(OBno)-OH. For coupling cycles following Asp-(OBno)-OH, the Fmoc protecting group was deprotected at room temperature. Chloroacetylation of the N-terminus was performed by treating the resin twice with chloroacetic anhydride (10 eq.) in NMP for 15 minutes at ambient temperature.

Step 2—Cleavage and Deprotection: The cleavage of the peptide from the solid support was achieved by treating the peptide-resin with reagent cocktail (87.5% TFA/5% water/2.5% TIS (triisopropylsilane)/2.5% Phenol: 15 mL/g of peptidyl-resin) for 1-2 hrs at room temperature or (60% TFA/5% TIS (triisopropylsilane)/35% DCM: 5 mL/g of peptidyl-resin) for 45 mins. at room temperature. The cleavage mixture was collected by filtration and the resin was washed with TFA. The peptide is precipitated in cold methyl tert-butyl ether, centrifuged (3800 rpm), and supernatant ether was removed. The peptide pellet was resuspended in cold methyl tert-butyl ether and centrifuged an additional two times. The precipitated peptide was then lyophilized under high vacuum overnight to give crude linear peptide.

Step 3—Peptide Cyclization: Crude linear peptide was dissolved at 1 mg/mL concentration in MeCN/H₂O (50:50) and DIPEA (10 eq) was added. The mixture was stirred at ambient temperature for 20 minutes. The pH was adjusted to ca. 5 with TFA and the mixture was freeze-dried.

Step 4—Peptide Purification: The crude cyclized peptide was purified using preparative HPLC using C4 Reverse Phase columns (Reprosil Gold, 120Å, 5 μm) and linear gradients of acetonitrile in water, both buffered with 0.1% TFA. The HPLC fractions containing a pure peptide product were pooled and lyophilized.

### Cyclic Peptide Sequence and Structure Examples

The macrocyclic peptides contain a thioether linkage between a cysteine sidechain and a —CH₂C(O) bond on the N-terminus. As illustrated in Table 1, representative cyclic peptides of the disclosure are described by a period-delimited sequence of amino acid residues and a structural representation. In the period-delimited sequence, substitution on amino acid residue sidechains is denoted in parentheses immediately following the amino acid residue that is substituted (see for example. Compounds 2, 3, 5 and 6 in Table 1 below). Reference macrocycle Compound 1a in Table 1 can be used as a reference to determine the structure for compounds solely listed by sequence as those compounds in Tables 2, 3, 4, 5, and 7. “*”s denote the location of the cyclized residues.

TABLE 1

| Compound | |
|---|---|
| # | Structure |
| 1a | 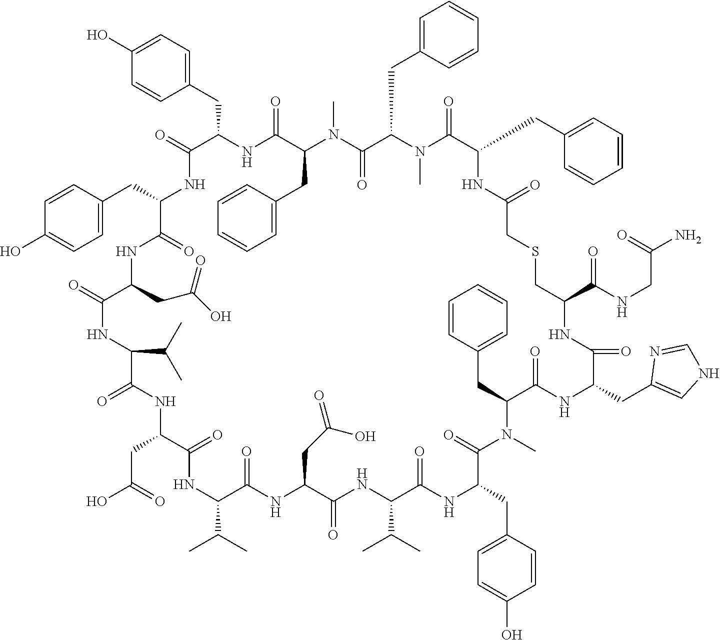 [SMILES: CC(C)[C@@H]1NC(=O)[C@H](CC(=O)O)NC(=O)[C@H](Cc2ccc(O)cc2)NC(=O)[C@H](Cc2ccc(O)cc2)NC(=O)[C@H](Cc2ccccc2)N(C)C(=O)[C@H](Cc2ccccc2)N(C)C(=O)[C@H](Cc2ccccc2)NC(=O)CSC[C@@H](C(=O)NCC(N)=O)NC(=O)[C@H](Cc2c[nH]cn2)NC(=O)[C@H](Cc2ccccc2)N(C)C(=O)[C@H](Cc2ccc(O)cc2)NC(=O)[C@H](C(C)C)NC(=O)[C@H](CC(=O)O)NC(=O)[C@H](C(C)C)NC(=O)[C@H](CC(=O)O)NC1=O] |
| | *F.NMeF.NMeF.Y.Y.D.V.D.V.D.V.Y.NMeF.H .*C.G-NH₂ (SEQ ID NO: 2) |
| 2a | 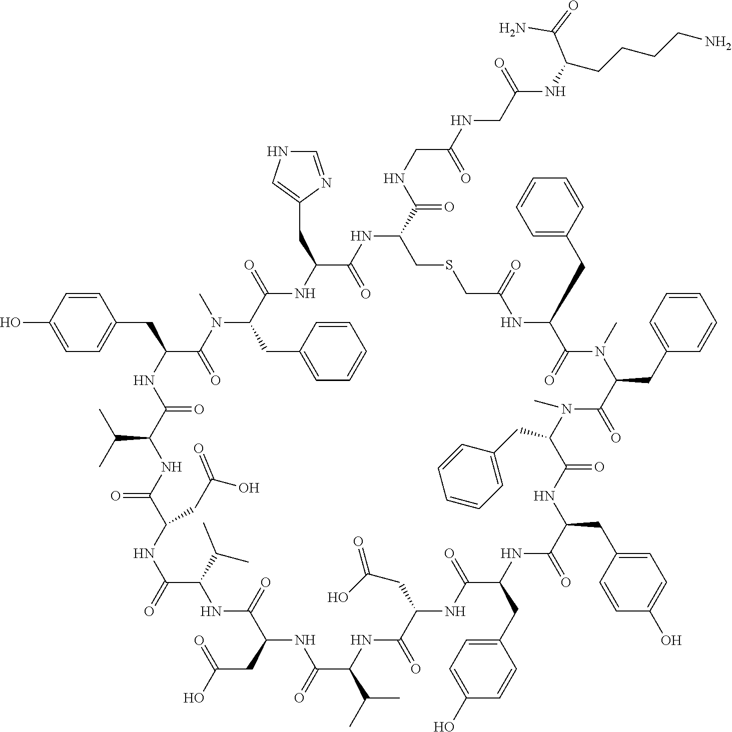 [SMILES: CC(C)[C@@H]1NC(=O)[C@H](CC(=O)O)NC(=O)[C@H](Cc2ccc(O)cc2)NC(=O)[C@H](Cc2ccc(O)cc2)NC(=O)[C@H](Cc2ccccc2)N(C)C(=O)[C@H](Cc2ccccc2)N(C)C(=O)[C@H](Cc2ccccc2)NC(=O)CSC[C@@H](C(=O)NCC(=O)NCC(=O)N[C@@H](CCCCN)C(N)=O)NC(=O)[C@H](Cc2c[nH]cn2)NC(=O)[C@H](Cc2ccccc2)N(C)C(=O)[C@H](Cc2ccc(O)cc2)NC(=O)[C@H](C(C)C)NC(=O)[C@H](CC(=O)O)NC(=O)[C@H](C(C)C)NC(=O)[C@H](CC(=O)O)NC1=O] |
| | *F.NMeF.NMeF.Y.Y.D.V.D.V.D.V.Y.NMeF.H.*C.G.G.K-NH₂ (SEQ ID NO: 3) |
| 3a | 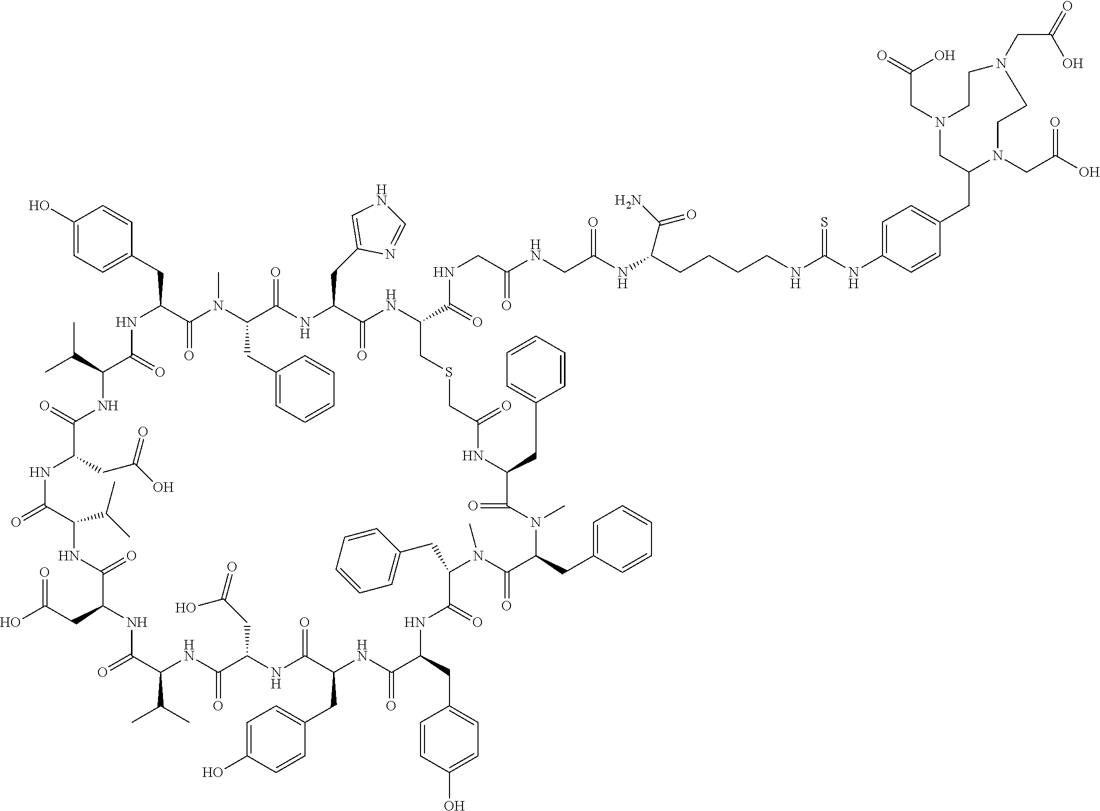 [SMILES: CC(C)[C@@H]1NC(=O)[C@H](CC(=O)O)NC(=O)[C@H](Cc2ccc(O)cc2)NC(=O)[C@H](Cc2ccc(O)cc2)NC(=O)[C@H](Cc2ccccc2)N(C)C(=O)[C@H](Cc2ccccc2)N(C)C(=O)[C@H](Cc2ccccc2)NC(=O)CSC[C@@H](C(=O)NCC(=O)NCC(=O)N[C@@H](CCCCNC(=S)Nc2ccc(CC3CN(CC(=O)O)CCN(CC(=O)O)CCN3CC(=O)O)cc2)C(N)=O)NC(=O)[C@H](Cc2c[nH]cn2)NC(=O)[C@H](Cc2ccccc2)N(C)C(=O)[C@H](Cc2ccc(O)cc2)NC(=O)[C@H](C(C)C)NC(=O)[C@H](CC(=O)O)NC(=O)[C@H](C(C)C)NC(=O)[C@H](CC(=O)O)NC1=O] |
| | *F.NMeF.Y.Y.D.V.D.V.D.V.Y.NMeF.H.*C.G.G.K(NOTA)-NH₂ (SEQ ID NO: 4) |
| 5a | 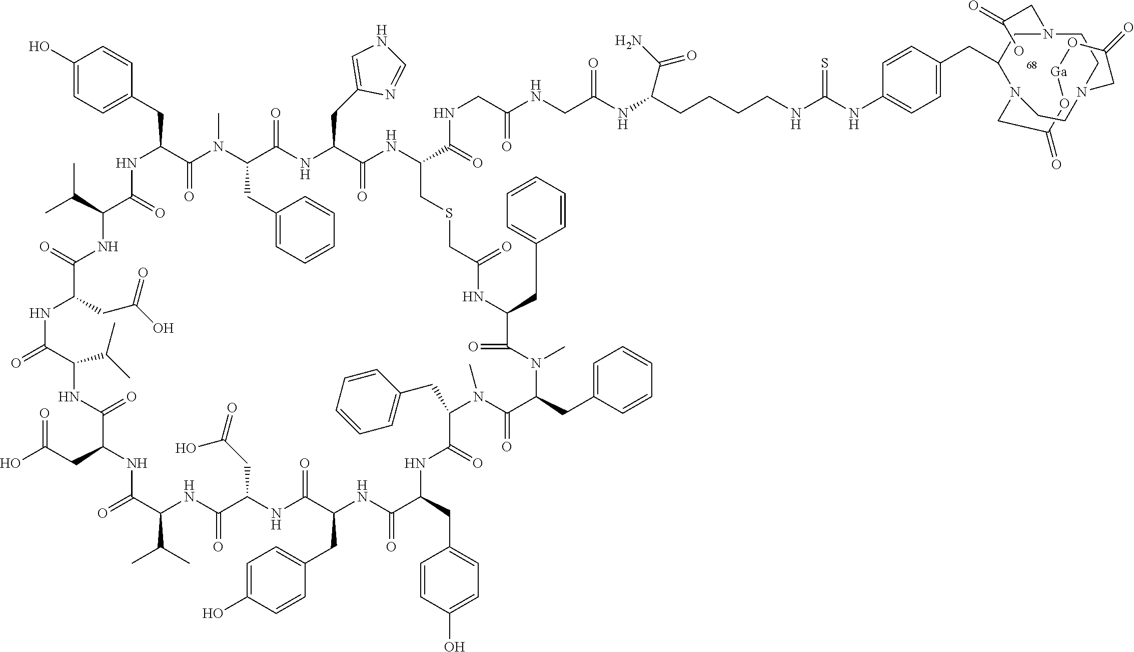 |
| | *F.NMeF.NMeF.Y.Y.D.V.D.V.D.V.Y.NMeF.H.*C.G.G.K(⁶⁸Ga-NOTA)-NH₂ (SEQ ID NO: 6) |
| 6a | 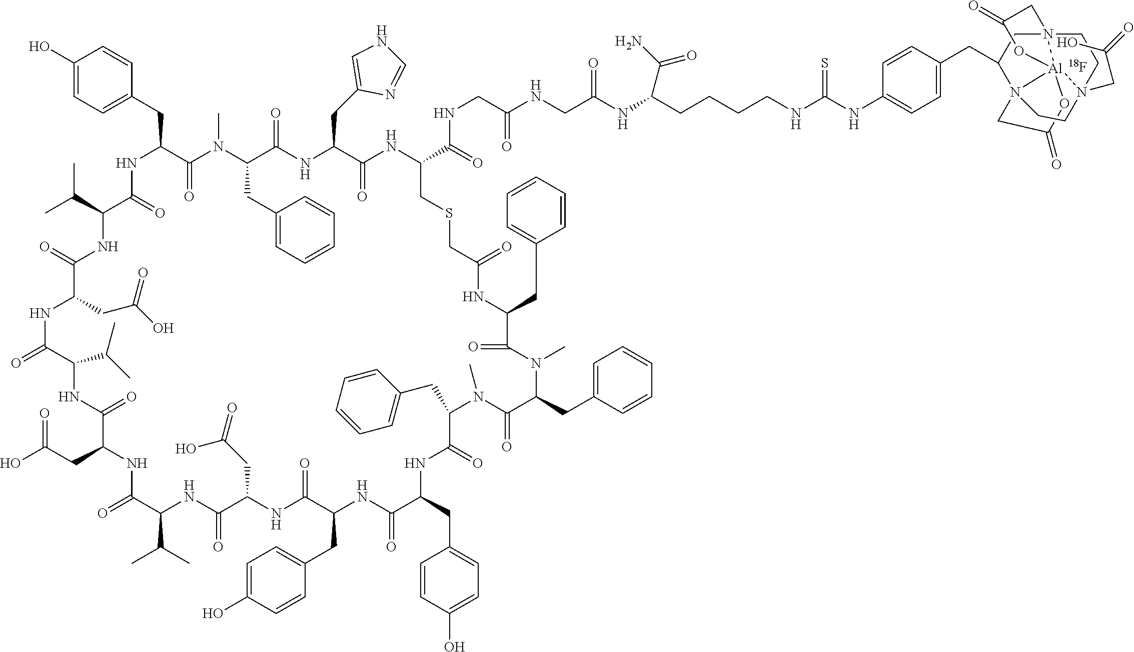 |
| | *F.NMeF.NMeF.Y.Y.D.V.D.V.D.V.Y.NMeF.H.*C.G.G.K(¹⁸F-AIF-NOTA)-NH₂ (SEQ ID NO: 7) |

Synthetic procedures for peptide derivatives not covered by general Protocol A or B:

### Example 1

### Preparation of Compound 1a (SEQ ID NO: 8)

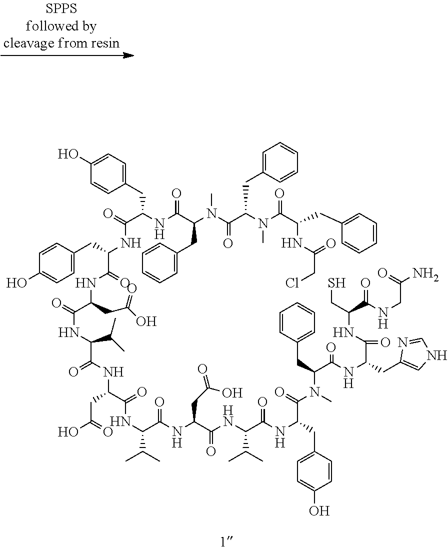 [SMILES: CC(C)[C@H](NC(=O)[C@H](CC(=O)O)NC(=O)[C@H](Cc1ccc(O)cc1)NC(=O)[C@H](Cc1ccc(O)cc1)NC(=O)[C@H](Cc1ccccc1)N(C)C(=O)[C@H](Cc1ccccc1)N(C)C(=O)[C@H](Cc1ccccc1)NC(=O)CCl)C(=O)N[C@@H](CC(=O)O)C(=O)N[C@H](C(=O)N[C@@H](CC(=O)O)C(=O)N[C@H](C(=O)N[C@@H](Cc1ccc(O)cc1)C(=O)N(C)[C@@H](Cc1ccccc1)C(=O)N[C@@H](Cc1c[nH]cn1)C(=O)N[C@@H](CS)C(=O)NCC(N)=O)C(C)C)C(C)C]

### Step A: Synthesis of 1″

The peptide sequence was assembled by solid phase synthesis on a Microwave Liberty Blue synthesizer (CEM, Matthews, NC). The synthesis was started using 526 mg (0.10 mmol) of Rink Amide Protide™ resin. 0.19 mmol/g (CEM). Each amino acid was coupled in 5-fold excess as a 0.2 M solution in DMF, which was activated using a solution 0.45 M HATU in DMF (1.0 mL, 0.45 mmol) and a solution of 1.0 M DIEA in NMP (1.0 mL, 1.0 mmol). Fmoc-deprotection steps were performed with 20% piperidine in DMF with for 60 seconds at 90° C. Single and double couplings were performed at 90° C. with 2 min coupling times. Acylation of secondary amino acids in the sequence was performed by double coupling. Chloroacetylation of the N-terminus was performed by treating the resin twice with chloroacetic anhydride (10 eq.) in NMP for 15 minutes at ambient temperature. Following solid-phase synthesis, the peptide was cleaved from the solid support by treating the peptide-resin with reagent cocktail (95% TFA/2.5% water/2.5% TIS (triisopropylsilane); 16 mL/g of peptidyl-resin) for 1 hour at room temperature. The cleavage mixture was collected by filtration, and the resin was washed with TFA. The peptide was precipitated in cold, centrifuged (3800 rpm), and supernatant ether was removed. The precipitated peptide is then lyophilized under high vacuum overnight to give 2″. MS: m/z=1077.7 [M+2H]/2

### Step B: Synthesis of 1a (SEQ ID NOS 8 and 2, Respectively, in Order of Appearance)

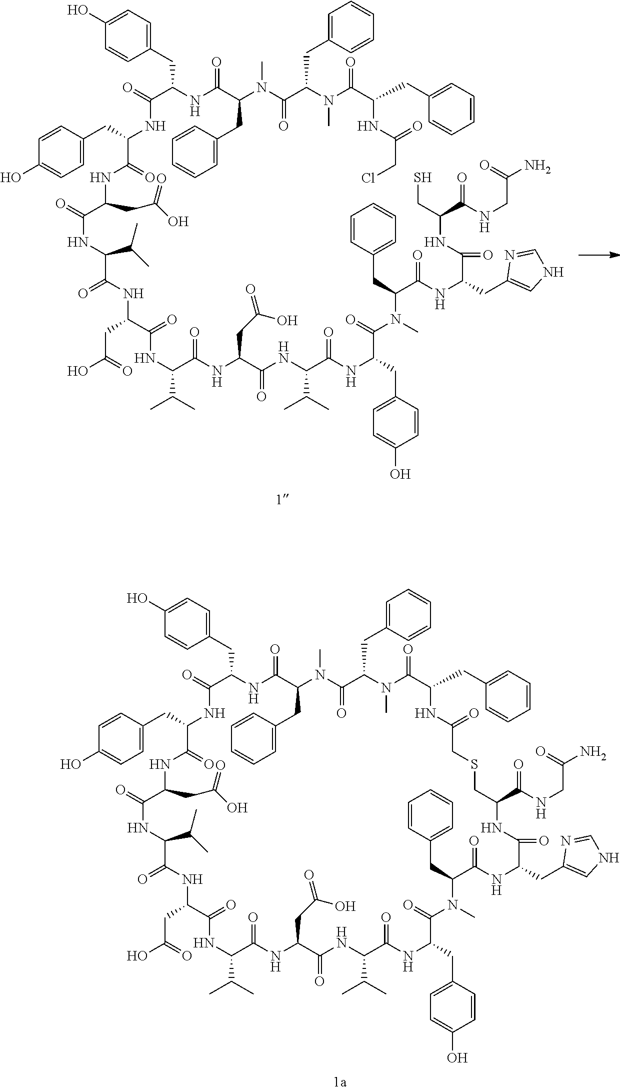

Compound 1″ (520 mg. 0.24 mmol) was dissolved in DMSO (1.67 mL) and added dropwise to a solution of DIEA (1.0 mmol, 174 μL) in DMSO (5.0 mL). The mixture was stirred at ambient temperature for 3 days. The sample was partially concentrated under a stream of nitrogen, then purified by mass-directed HPLC using C18 reverse phase column (2 cm×5 cm. 5 μm) and 40-60% acetonitrile in water, both buffered with 0.1% TFA. The HPLC fractions containing a pure peptide product were pooled and lyophilized to give 1a. MS: m/z=1059.4 [M+2H]/2

### Example 2

### Preparation of Compound 2a (SEQ ID NO: 9)

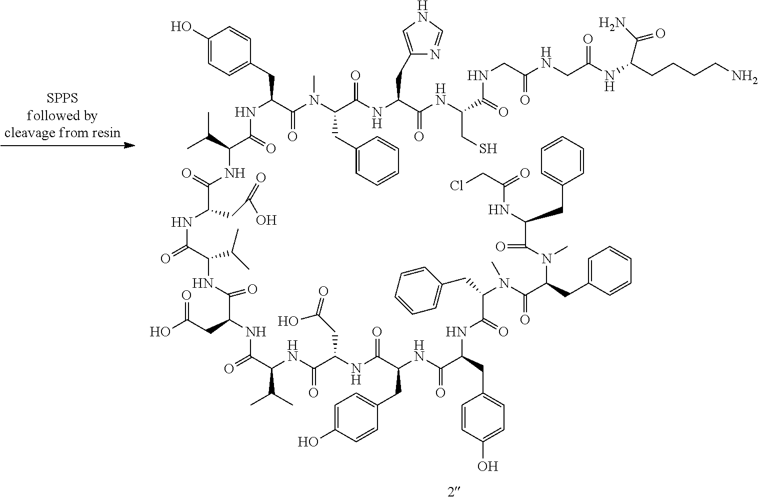 [SMILES: CC(C)[C@H](NC(=O)[C@H](CC(=O)O)NC(=O)[C@H](Cc1ccc(O)cc1)NC(=O)[C@H](Cc1ccc(O)cc1)NC(=O)[C@H](Cc1ccccc1)N(C)C(=O)[C@H](Cc1ccccc1)N(C)C(=O)[C@H](Cc1ccccc1)NC(=O)CCl)C(=O)N[C@@H](CC(=O)O)C(=O)N[C@H](C(=O)N[C@@H](CC(=O)O)C(=O)N[C@H](C(=O)N[C@@H](Cc1ccc(O)cc1)C(=O)N(C)[C@@H](Cc1ccccc1)C(=O)N[C@@H](Cc1c[nH]cn1)C(=O)N[C@@H](CS)C(=O)NCC(=O)NCC(=O)N[C@@H](CCCCN)C(N)=O)C(C)C)C(C)C]

### Step A: Synthesis of 2″

The peptide sequence was assembled by solid phase synthesis on a Microwave Liberty Blue synthesizer (CEM, Matthews, NC). The synthesis was started using 526 mg (0.10 mmol) of Rink Amide Protidel™ resin. 0.19 mmol/g (CEM). Each amino acid was coupled in 5-fold excess as a 0.2 M solution in DMF, which was activated using a solution 0.45 M HATU in DMF (1.0 mL, 0.45 mmol) and a solution of 1.0 M DIEA in NMP (1.0 mL, 1.0 mmol). Fmoc-deprotection steps were performed with 20% piperidine in DMF with for 60 seconds at 90° C. Single and double couplings were performed at 90° C. with 2 min coupling times. Acylation of secondary amino acids in the sequence was performed by double coupling. Chloroacetylation of the N-terminus was performed by treating the resin twice with chloroacetic anhydride (10 eq.) in NMP for 15 minutes at ambient temperature. Following solid-phase synthesis, the peptide was cleaved from the solid support by treating the peptide-resin with reagent cocktail (95% TFA/2.5% water/2.5% TIS (triisopropylsilane); 16 mL/g of peptidyl-resin) for 1 hour at room temperature. The cleavage mixture was collected by filtration and the resin was washed with TFA. The peptide was precipitated in cold, centrifuged (3800 rpm), and supernatant ether was removed. The precipitated peptide was then lyophilized under high vacuum overnight to give 2″. MS: m/z=1169.6 [M+2H]/2

### Step B: Synthesis of 2 (SEQ ID NOS 9 and 3, Respectively, in Order of Appearance)

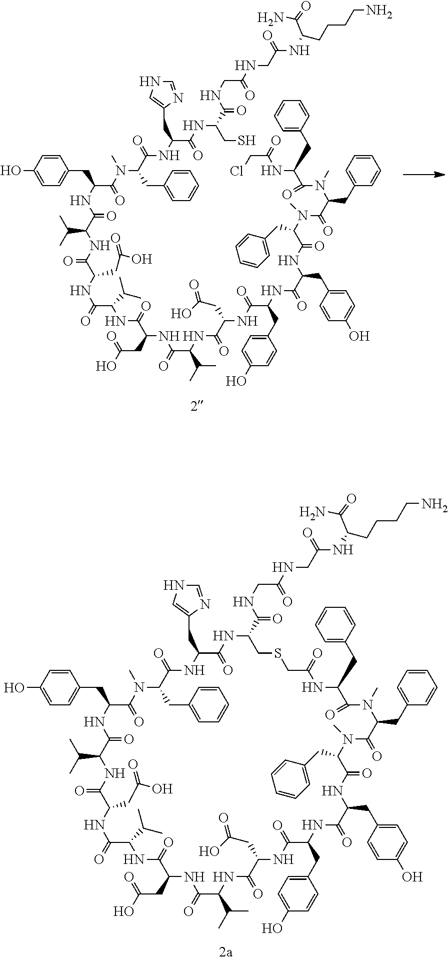

Compound 2″ (240 mg, 0.089 mmol) was dissolved in DMSO (1.74 mL) to a final concentration of 0.05 M. This solution was added dropwise to a solution of DIPEA (5 eq, 87 μL) in DMSO (6.96 mL). The mixture was stirred at ambient temperature for 2 hours, then additional DIPEA (5 eq, 87 μL) was added. The sample was partially concentrated under a stream of nitrogen, then purified by mass-directed HPLC using C18 reverse phase column (2 cm×5 cm. 5 μm) and elution with 40-60% acetonitrile in water, both buffered with 0.1% TFA. The HPLC fractions containing a pure peptide product were pooled and lyophilized to give 2a. MS: m/z=1151.9 [M+2H]/2.

The compounds in Table 2 of the present invention were made using the method described in Example 2 and substituting the appropriate reactants and/or reagents:

TABLE 2

| | | SEQ | Observed |
|---|---|---|---|
| Compound | | ID | m/z |
| # | Sequence | NO: | [M+ 2H]/2 |
| 9a | *F.NMeF.A.Y.Y.D.V.D.V.D.V.Y.NMeF.H.*C.G.G.K-NH₂ | 11 | 1107.1 |
| 10a | *F.NMeF.NMeF.Y.A.D.V.D.V.D.V.Y.NMeF.H.*C.G.K-NH₂ | 12 | 1077.8 |
| 11a | *F.NMeF.NMeF.Y.A.D.V.D.V.D.V.Y.NMeF.H.*C.G.K.K-NH₂ | 13 | 1141.7 |
| 12a | *F.NMeF.NMeF.Y.Y.D.V.D.V.D.V.Y.NMeF.H.*C.G.K-NH₂ | 14 | 1123.7 |
| 13a | *F.NMeF.NMeF.Y.Y.D.V.D.V.D.V.Y.NMeF.H.*C.G.K.K-NH₂ | 15 | 1187.9 |

### Example 3

### Preparation of Compound 3a (SEQ ID NO: 4)

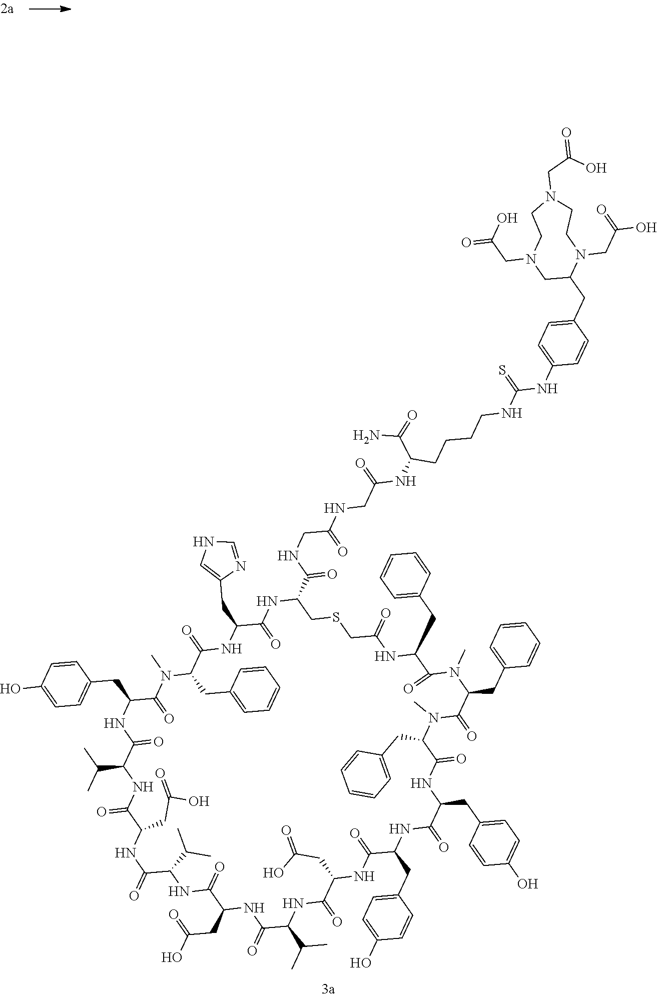 [SMILES: CC(C)[C@@H]1NC(=O)[C@H](CC(=O)O)NC(=O)[C@H](Cc2ccc(O)cc2)NC(=O)[C@H](Cc2ccc(O)cc2)NC(=O)[C@H](Cc2ccccc2)N(C)C(=O)[C@H](Cc2ccccc2)N(C)C(=O)[C@H](Cc2ccccc2)NC(=O)CSC[C@@H](C(=O)NCC(=O)NCC(=O)N[C@@H](CCCCNC(=S)Nc2ccc(CC3CN(CC(=O)O)CCN(CC(=O)O)CCN3CC(=O)O)cc2)C(N)=O)NC(=O)[C@H](Cc2c[nH]cn2)NC(=O)[C@H](Cc2ccccc2)N(C)C(=O)[C@H](Cc2ccc(O)cc2)NC(=O)[C@H](C(C)C)NC(=O)[C@H](CC(=O)O)NC(=O)[C@H](C(C)C)NC(=O)[C@H](CC(=O)O)NC1=O]

A solution of commercially available 2,2′,2″-(2-(4-isothiocyanatobenzyl)-1,4,7-triazonane-1,4,7-triyl)triacetic acid (40 mg, 0.039 mmol) in DMSO (1.5 ml) and DIPEA (14 μL, 0.78 mmol) was added to a mixture of peptide 2 (60 mg, 0.026 mmol) and DIPEA (14 μL, 0.78 mmol) in DMSO (1.5 ml). After stirring at room temperature for 4 h, the pH was adjusted to ca. 5 with TFA. The crude material was purified using preparative HPLC using C4 Reverse Phase columns (Delta-Pak Double Cartridge 25×100 mm, 300Å, 5 μm) eluting 15-35% acetonitrile in water, both buffered with 0.100 TFA. The HPLC fractions containing a pure peptide product were pooled and lyophilized. MS: m/z=1377.1 [M+2H]/2.

The compounds in Table 3 of the present invention were made using the method described in this example and substituting the appropriate reactants and/or reagents:

TABLE 3

| | | Observed |
|---|---|---|
| Compound | | m/z |
| # | Sequence | [M + 2H]/2 |
| 8a | *F.NMeF.NMeF.Y.Y.D.V.D.A.D.V.Y.NMeF.H.*C.G.G.K | 1363.3 |
| | (NOTA)-NH₂ (SEQ ID NO: 17) | |

### Example 4

### Preparation of Compound 4a (SEQ ID NO: 5)

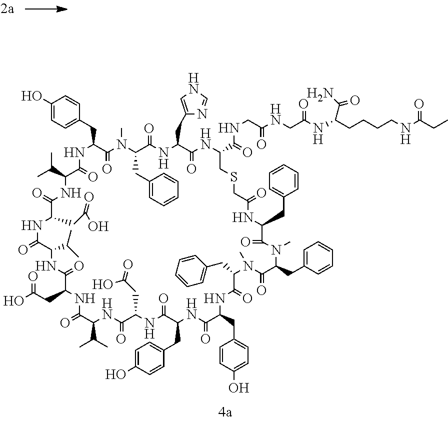 [SMILES: CCC(=O)NCCCC[C@H](NC(=O)CNC(=O)CNC(=O)[C@@H]1CSCC(=O)N[C@@H](Cc2ccccc2)C(=O)N(C)[C@@H](Cc2ccccc2)C(=O)N(C)[C@@H](Cc2ccccc2)C(=O)N[C@@H](Cc2ccc(O)cc2)C(=O)N[C@@H](Cc2ccc(O)cc2)C(=O)N[C@@H](CC(=O)O)C(=O)N[C@@H](C(C)C)C(=O)N[C@@H](CC(=O)O)C(=O)N[C@@H](C(C)C)C(=O)N[C@@H](CC(=O)O)C(=O)N[C@@H](C(C)C)C(=O)N[C@@H](Cc2ccc(O)cc2)C(=O)N(C)[C@@H](Cc2ccccc2)C(=O)N[C@@H](Cc2c[nH]cn2)C(=O)N1)C(N)=O]

To a solution of propionic acid (1.3 mg, 0.017 mmol) in DMF (0.5 mL), HATU (5.0 mg, 0.013 mmol) and DIPEA (4.5 μL, 0.026 mmol) were added. After 5 minutes, the mixture was added to a solution of peptide 2 (20 mg, 0.0087 mmol) in DMF (2.5 mL) and stirred for 15 minutes at ambient temperature. The pH was adjusted to ca. 5 with TFA. The crude material was purified using preparative HPLC using C4 Reverse Phase columns (Reprosil Gold, 120Å, 5 μm) or Phenomenex Luna C18 5u 100A 250*21.2 mm and linear gradients of acetonitrile in water, both buffered with 0.10% TFA. The HPLC fractions containing a pure peptide product were pooled and lyophilized. MS: m/z 1179.7 [M+2H]/2.

### Example 5

### Preparation of Compound 5a (SEQ ID NO: 6)

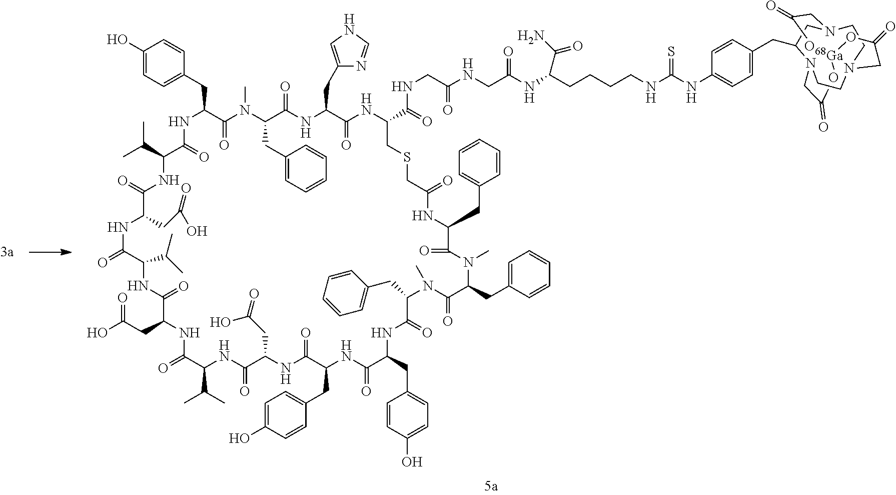

[⁶⁸Ga]GaCl₃ was eluted from a IGG100 Gallium-68 generator (Eckert & Ziegler) using 5 ml of 0.1 M HCl and passed through a Phenomenex Strata-XC cartridge. [⁶⁸Ga]GaCl₃ was then eluted from the cartridge with 0.5 ml of 98% acetone (in 0.02 M HCl). For labeling, compound 3 (50 μg, 20 nmol) was dissolved in 500 L of HEPES buffer (0.1 M, pH 4.1) and 0.26 GBq of [⁶⁸Ga]GaCl₃ were mixed and incubated at 70° C. for 15 min. Radiochemical purity was monitored by radio-iTLC or radio-HPLC on an Onyx Monolithic C18 column (100 mm×3 mm,) eluted with a gradient from 5% acetonitrile in aqueous (0.1% HCO₂H) to 90% acetonitrile at a flow rate of 1.5 ml/min over 7 min.

### Example 6

### Preparation of Compound 6a (SEQ ID NO: 7)

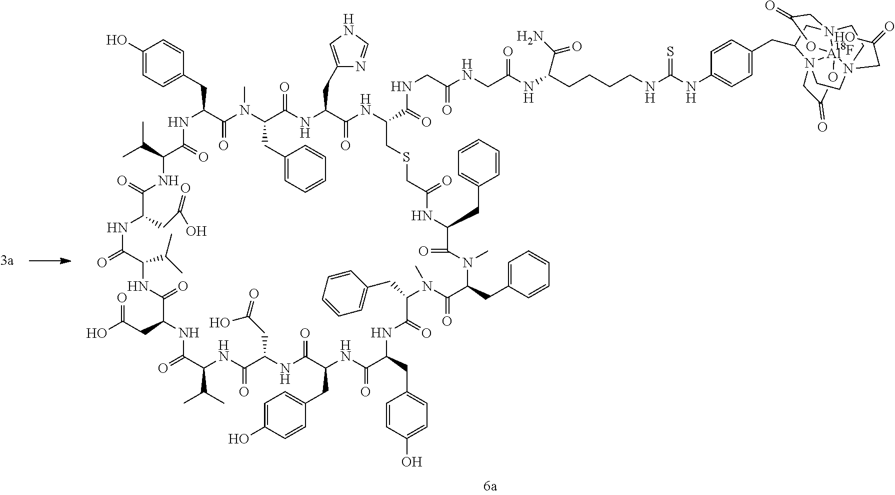

¹⁸F⁻ (7.4GBq) was released from QMA cartridge using 0.4 mL of KHCO₃ (0.4 M) into a vial containing 125 μL sodium acetate (0.1M, pH 4), 25 μL AlCl₃ (2 mM in 0.1 M sodium acetate) and 10 μL acetic acid. The mixture was kept at room temperature for 2 min. Compound 3 (0.4 mg, 0.17 μmol), dissolved in a mixture of 75 L 0.1M sodium acetate (pH 4) and 75 μL acetonitrile, was then added to the mixture above. After heating at 100° C. for 15 min. 700 μL of deionized (DI) water was added to the reaction solution, and then loaded into HPLC for purification. For this, a Phenomenex Gemini C18 column (10 mm I.D.×150 mm) was eluted at a flow rate of 5 mL/min with a gradient consisting of 0.1% TFA both in water and acetonitrile. The proportion of acetonitrile was linearly increased from 20% to 70% in 15 min. The solvent in the pooled HPLC fractions containing the radiolabeled peptides was partially concentrated under reduced pressure. Radiochemical purity was monitored by radio-iTLC or radio-HPLC on an Onyx Monolithic C18 column (100 mm×3 mm,) eluted with a gradient from 5% acetonitrile in aqueous (0.1% HCO₂H) to 90% acetonitrile at a flow rate of 1.5 ml/min over 7 min.

The compound in Table 4 of the present invention was made using the method described in this example and substituting the appropriate reactants and/or reagents:

TABLE 4

| Compound # | Sequence |
|---|---|
| 7a | *F.NMeF.NMeF.Y.Y.D.V.D.A.D.V.Y.NMeF.H.*C.G.G.K(¹⁸F-AIF- |
| | NOTA)-NH₂ (SEQ ID NO: 19) |

### Example 7

### Preparation of Compound 26a (SEQ ID NO: 20)

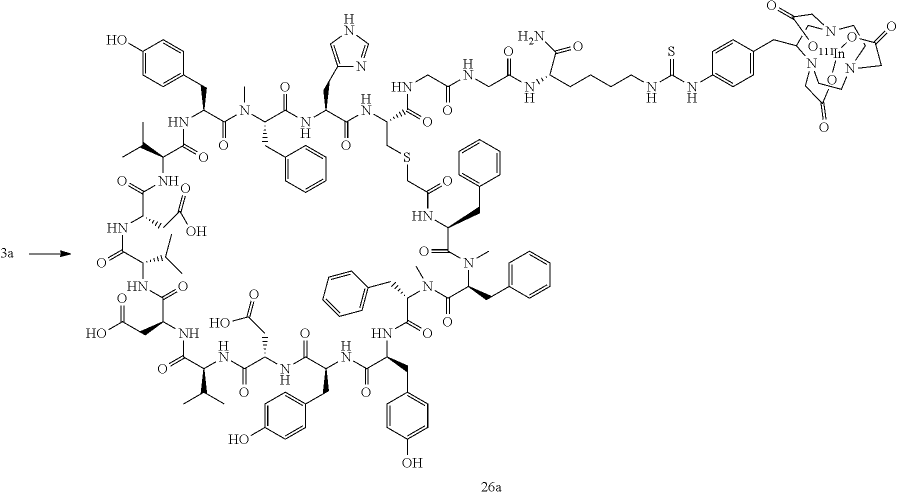

[¹¹¹In]-Indium chloride (Jubilant Radiopharmacies, Folcroft, PA) (100 μl, 52 MBq) was added to a solution containing compound 3 (50 μg, 17 nmol) in HEPES buffer (0.1 M, pH 4.1, 50 μl) diluted in HCl (0.05 N. 800 μl). After incubation at 95° C. for 30 min, radiochemical purity was monitored by radio-iTLC and the sample used without further purification.

### Example 8

### Preparation of Compound 27a (SEQ ID NO: 21)

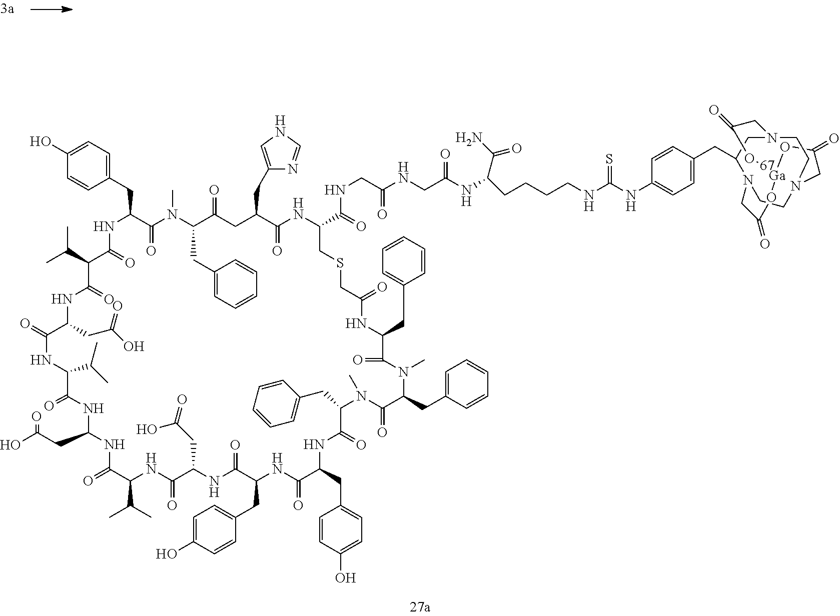

[⁶⁷Ga]-Gallium chloride (Lantheus Medical Imaging, North Billerica, MA) (300 μl, 68 MBq) was added to a solution containing compound 3 (50 μg, 18.2 nmol) in HEPES buffer (0.1 M, pH 6-6.5, 50 μl) diluted in HEPES buffer (0.1 M, pH 7, 270 μl). After incubation at 37° C. for 30 min, radiochemical purity was monitored by radio-iTLC and the sample used without further purification.

The cyclic peptides in Table 5 were prepared according to Protocol A or Protocol B outlined and/or exemplified in the disclosure.

TABLE 5

| | | Observed | Granzyme |
|---|---|---|---|
| Compd | | m/z | B SPR |
| # | Sequence | [M + 2H]/2 | Kd (nm) |
| 1a | *F.NMeF.NMeF.Y.Y.D.V.D.V.D.V.Y.NMeF.H.*C.G-NH₂ | 1059.6 | 0.5 |
| | (SEQ ID NO: 2) | | |
| 8a | *F.NMeF.NMeF.Y.Y.D.V.D.V.D.V.Y.NMeF.A.*C.G-NH₂ | 1026.2 | 0.8 |
| | (SEQ ID NO: 22) | | |
| 9a | *F.NMeF.NMeF.Y.A.D.V.D.V.D.V.Y.NMeF.H.*C.G-NH₂ | 1014.3 | 0.8 |
| | (SEQ ID NO: 23) | | |
| 10a | *F.NMeF.NMeF.Y.Y.D.V.D.V.D.V.Y.NMeF.H.*C.G.G.K-NH₂ | 1152.0 | 1.1 |
| | (SEQ ID NO: 3) | | |
| 11a | *F.NMeF.NMeF.Y.Y.D.V.D.A.D.V.Y.NMeF.H.*C.G-NH₂ | 1046.2 | 4.7 |
| | (SEQ ID NO: 24) | | |
| 12a | *F.NMeF.NMeF.Y.Y.D.V.D.V.D.A.Y.NMeF.H.*C.G-NH₂ | 1045.6 | 10.7 |
| | (SEQ ID NO: 25) | | |
| 13a | *F.NMeF.A.Y.Y.D.V.D.V.D.V.Y.NMeF.H.*C.G-NH₂ | 1014.1 | 68 |
| | (SEQ ID NO: 26) | | |
| 14a | *A.NMeF.NMeF.Y.Y.D.V.D.V.D.V.Y.NMeF.H.*C.G-NH₂ | 1022.4 | 165.0 |
| | (SEQ ID NO: 27) | | |
| 15a | *F.NMeF.A.Y.Y.D.V.D.V.D.V.Y.NMeF.H.*C.G.G.K-NH₂ | 1107.1 | 188 |
| | (SEQ ID NO: 11) | | |
| 16a | *F.NMeF.NMeF.Y.Y.D.A.D.V.D.V.Y.NMeF.H.*C.G-NH₂ | 1046.3 | 288.0 |
| | (SEQ ID NO: 28) | | |
| 17a | *F.NMeF.NMeF.Y.Y.D.V.D.V.D.V.A.NMeF.H.*C.G-NH₂ | 1014.3 | 453.0 |
| | (SEQ ID NO: 29) | | |
| 18a | *F.NMeF.NMeF.Y.Y.D.V.A.V.D.V.Y.NMeF.H.*C.G-NH₂ | 1038.2 | 548.0 |
| | (SEQ ID NO: 30) | | |
| 19a | *F.NMeF.NMeF.A.Y.D.V.D.V.D.V.Y.NMeF.H.*C.G-NH₂ | 1014.5 | 752.0 |
| | (SEQ ID NO: 31) | | |
| 20a | *F.NMeF.NMeF.Y.A.D.V.D.V.D.V.Y.NMeF.H.*C.G.K-NH₂ | 1077.8 | 1.0 |
| | (SEQ ID NO: 12) | | |
| 21a | *F.NMeF.NMeF.Y.A.D.V.D.V.D.V.Y.NMeF.H.*C.G.K.K-NH₂ | 1141.7 | 2.1 |
| | (SEQ ID NO: 13) | | |
| 22a | *F.NMeF.NMeF.Y.Y.D.V.D.V.D.V.Y.NMeF.H.*C.G.K-NH₂ | 1123.7 | 2.6 |
| | (SEQ ID NO: 14) | | |
| 23a | *F.NMeF.NMeA.Y.Y.D.V.D.V.D.V.Y.NMeF.H.*C.G-NH₂ | 1021.7 | 5.4 |
| | (SEQ ID NO: 32) | | |
| 24a | *F.NMcA.NMcF.Y.Y.D.V.D.V.D.V.Y.NMcF.H.*C.G-NH₂ | 1021.8 | 199.4 |
| | (SEQ ID NO: 33) | | |
| 25a | *F.NMeF.NMeF.Y.Y.D.V.D.V.D.V.Y.NMeF.H.*C.G.K.K-NH₂ | 1187.9 | 0.1 |
| | (SEQI D NO: 15) | | |

The cyclic peptides of the disclosure were prepared according to Protocol A or Protocol B outlined and/or exemplified in the disclosure.

Amino acid linear sequence for parent peptides 1-27 are listed in Table 6.

TABLE 6

| Compd | |
|---|---|
| # | Linear Peptide Sequence |
| 1 | ClAc.F.NMeF.NMeF.Y.Y.D.V.D.V.D.V.Y.NMeF.H.C.G-NH₂ (SEQ ID NO: 8) |
| 2 | ClAc.F.NMeF.NMeF.Y.Y.D.V.D.V.D.V.Y.NMeF.H.C.G.G.K-NH₂ (SEQ ID NO: 9) |
| 3 | ClAc.F.NMeF.NMeF.Y.Y.D.V.D.V.D.V.Y.NMeF.H.C.G.G.K(NOTA)-NH₂ (SEQ ID NO: |
| | 34) |
| 4 | ClAc.F.NMeF.NMeF.Y.Y.D.V.D.V.D.V.Y.NMeF.H.C.G.G.K(Prop)-NH₂ (SEQ ID NO: |
| | 35) |
| 5 | ClAc.F.NMeF.NMeF.Y.Y.D.V.D.V.D.V.Y.NMeF.H.C.G.G.K(⁶⁸Ga-NOTA)-NH₂ (SEQ |
| | ID NO: 36) |
| 6 | ClAc.F.NMeF.NMeF.Y.Y.D.V.D.V.D.V.Y.NMeF.H.C.G.G.K(¹⁸F-AlF-NOTA)-NH₂ |
| | (SEQ ID NO: 37) |
| 7 | ClAc.F.NMeF.NMeF.Y.Y.D.V.D.A.D.V.Y.NMeF.H.C.G.G.K(¹⁸F-AlF-NOTA)-NH₂ |
| | (SEQ ID NO: 38) |
| 8 | ClAc.F.NMeF.NMeF.Y.Y.D.V.D.A.D.V.Y.NMeF.H.C.G.G.K(NOTA)-NH₂ (SEQ ID NO: |
| | 39) |
| 9 | ClAc.F.NMeF.A.Y.Y.D.V.D.V.D.V.Y.NMeF.H.C.G.G.K-NH₂ (SEQ ID NO: 40) |
| 10 | ClAc.F.NMeF.NMeF.Y.A.D.V.D.V.D.V.Y.NMeF.H.C.G.K-NH₂ (SEQ ID NO: 41) |
| 11 | ClAc.F.NMeF.NMeF.Y.A.D.V.D.V.D.V.Y.NMeF.H.C.G.K.K-NH₂ (SEQ ID NO: 42) |
| 12 | ClAc.F.NMeF.NMeF.Y.Y.D.V.D.V.D.V.Y.NMeF.H.C.G.K-NH₂ (SEQ ID NO: 43) |
| 13 | ClAc.F.NMeF.NMeF.Y.Y.D.V.D.V.D.V.Y.NMeF.H.C.G.K.K-NH₂ (SEQ ID NO: 44) |
| 14 | ClAc.F.NMeF.NMeF.Y.Y.D.V.D.V.D.V.Y.NMeF.A.C.G-NH₂ (SEQ ID NO: 45) |
| 15 | ClAc.F.NMeF.NMeF.Y.A.D.V.D.V.D.V.Y.NMeF.H.C.G-NH₂ (SEQ ID NO: 46) |
| 16 | ClAc.F.NMeF.NMeF.Y.Y.D.V.D.A.D.V.Y.NMeF.H.C.G-NH₂ (SEQ ID NO: 47) |
| 17 | ClAc.F.NMeF.NMeF.Y.Y.D.V.D.V.D.A.Y.NMeF.H.C.G-NH₂ (SEQ ID NO: 48) |
| 18 | ClAc.F.NMeF.A.Y.Y.D.V.D.V.D.V.Y.NMeF.H.C.G-NH₂ (SEQ ID NO: 49) |
| 19 | CIAc.A.NMeF.NMeF.Y.Y.D.V.D.V.D.V.Y.NMeF.H.C.G-NH₂ (SEQ ID NO: 50) |
| 20 | ClAc.F.NMeF.NMeF.Y.Y.D.A.D.V.D.V.Y.NMeF.H.C.G-NH₂ (SEQ ID NO: 51) |
| 21 | ClAc.F.NMeF.NMeF.Y.Y.D.V.D.V.D.V.A.NMeF.H.C.G-NH₂ (SEQ ID NO: 52) |
| 22 | ClAc.F.NMeF.NMeF.Y.Y.D.V.A.V.D.V.Y.NMeF.H.C.G-NH₂ (SEQ ID NO: 53) |
| 23 | ClAc.F.NMeF.NMeF.A.Y.D.V.D.V.D.V.Y.NMeF.H.C.G-NH₂ (SEQ ID NO: 54) |
| 24 | ClAc.F.NMeF.NMeA.Y.Y.D.V.D.V.D.V.Y.NMeF.H.C.G-NH₂ (SEQ ID NO: 55) |
| 25 | ClAc.F.NMeA.NMeF.Y.Y.D.V.D.V.D.V.Y.NMeF.H.C.G-NH₂ (SEQ ID NO: 16) |
| 26 | ClAc.F.NMeF.NMeF.Y.Y.D.V.D.V.D.V.Y.NMeF.H.C.G.G.K(¹¹¹In-NOTA)-NH₂ (SEQ |
| | ID NO: 18) |
| 27 | ClAc.F.NMeF.NMeF.Y.Y.D.V.D.V.D.V.Y.NMeF.H.C.G.G.K(⁶⁷Ga-NOTA)-NH₂ (SEQ |
| | ID NO: 10) |

Amino acid sequence for macrocyclic peptides 1a-27a are listed in Table 7. The asterisks (*) depict where cyclization (i.e., point of amino acid linkage in the molecule) exists, i.e., between amino acid alanine (A) residue, or phenylalanine (F) reside and sulfur on amino acid cysteine (C) residue, and where the amino acid-NH₂″ (e.g., “G-NH₂” or “K—NH₂”) contains the C-terminal carboxamide. See Compound 1a, for example, as depicted below.

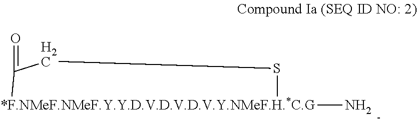

TABLE 7

| Compd# | Macrocyclic Peptide Sequence |
|---|---|
| 1a | *F.NMeF.NMeF.Y.Y.D.V.D.V.D.V.Y.NMeF.H.*C.G-NH₂ (SEQ ID NO: 2) |
| 2a | *F.NMeF.NMeF.Y.Y.D.V.D.V.D.V.Y.NMeF.H.*C.G.G.K-NH₂ (SEQ ID NO: 3) |
| 3a | *F.NMeF.NMeF.Y.Y.D.V.D.V.D.V.Y.NMeF.H.*C.G.G.K(NOTA)-NH₂ (SEQ ID |
| | NO: 4) |
| 4a | *F.NMeF.NMeF.Y.Y.D.V.D.V.D.V.Y.NMeF.H.*C.G.G.K(Prop)-NH₂ (SEQ ID |
| | NO: 5) |
| 5a | *F.NMeF.NMeF.Y.Y.D.V.D.V.D.V.Y.NMeF.H.*C.G.G.K(⁶⁸Ga-NOTA)-NH₂ |
| | (SEQ ID NO: 6) |
| 6a | *F.NMeF.NMeF.Y.Y.D.V.D.V.D.V.Y.NMeF.H.*C.G.G.K(¹⁸F-AlF-NOTA)-NH₂ |
| | (SEQ ID NO: 7) |
| 7a | *.F.NMeF.NMeF.Y.Y.D.V.D.A.D.V.Y.NMeF.H.*C.G.G.K(¹⁸F-AlF-NOTA)-NH₂ |
| | (SEQ ID NO: 19) |
| 8a | *F.NMeF.NMeF.Y.Y.D.V.D.A.D.V.Y.NMeF.H.*C.G.G.K(NOTA)-NH₂ (SEQ ID |
| | NO: 17) |
| 9a | *F.NMeF.A.Y.Y.D.V.D.V.D.V.Y.NMeF.H.*C.G.G.K-NH₂ (SEQ ID NO: 11) |
| 10a | *F.NMeF.NMeF.Y.A.D.V.D.V.D.V.Y.NMeF.H.*C.G.K-NH₂ (SEQ ID NO: 12) |
| 11a | *F.NMeF.NMeF.Y.A.D.V.D.V.D.V.Y.NMeF.H.*C.G.K.K-NH₂ (SEQ ID NO: |
| | 13) |
| 12a | *F.NMeF.NMeF.Y.Y.D.V.D.V.D.V.Y.NMeF.H.*C.G.K-NH₂ (SEQ ID NO: 14) |
| 13a | *F.NMeF.NMeF.Y.Y.D.V.D.V.D.V.Y.NMeF.H.*C.G.K.K-NH₂ (SEQ ID NO: |
| | 15) |
| 14a | *F.NMeF.NMeF.Y.Y.D.V.D.V.D.V.Y.NMeF.A.*C.G-NH₂ (SEQ ID NO: 22) |
| 15a | *F.NMeF.NMeF.Y.A.D.V.D.V.D.V.Y.NMeF.H.*C.G-NH₂ (SEQ ID NO: 23) |
| 16a | *F.NMeF.NMeF.Y.Y.D.V.D.A.D.V.Y.NMeF.H.*C.G-NH₂ (SEQ ID NO: 24) |
| 17a | *F.NMeF.NMeF.Y.Y.D.V.D.V.D.A.Y.NMeF.H.*C.G-NH₂ (SEQ ID NO: 25) |
| 18a | *F.NMeF.A.Y.Y.D.V.D.V.D.V.Y.NMeF.H.*C.G-NH₂ (SEQ ID NO: 26) |
| 19a | *A.NMeF.NMeF.Y.Y.D.V.D.V.D.V.Y.NMeF.H.*C.G-NH₂ (SEQ ID NO: 27) |
| 20a | *F.NMeF.NMeF.Y.Y.D.A.D.V.D.V.Y.NMeF.H.*C.G-NH₂ (SEQ ID NO: 28) |
| 21a | *F.NMeF.NMeF.Y.Y.D.V.D.V.D.V.A.NMeF.H.*C.G-NH₂ (SEQ ID NO: 29) |
| 22a | *F.NMeF.NMeF.Y.Y.D.V.A.V.D.V.Y.NMeF.H.*C.G-NH₂ (SEQ ID NO: 30) |
| 23a | *F.NMeF.NMeF.A.Y.D.V.D.V.D.V.Y.NMeF.H.*C.G-NH₂ (SEQ ID NO: 31) |
| 24a | *F.NMeF.NMeA.Y.Y.D.V.D.V.D.V.Y.NMeF.H.*C.G-NH₂ (SEQ ID NO: 32) |
| 25a | *F.NMeA.NMeF.Y.Y.D.V.D.V.D.V.Y.NMeF.H.*C.G-NH₂ (SEQ ID NO: 33) |
| 26a | *.F.NMeF.NMeF.Y.Y.D.V.D.V.D.V.Y.NMeF.H.*C.G.G.K(¹¹¹In-NOTA)-NH₂ |
| | (SEQ ID NO: 20) |
| 27a | *.F.NMeF.NMeF.Y.Y.D.V.D.V.D.V.Y.NMeF.H.*C.G.G.K(⁶⁷Ga-NOTA)-NH₂ |
| | (SEQ ID NO: 21) |

### Biological Assays:

### Surface Plasmon Resonance (SPR)

The kinetics of test peptide binding to human Granzyme B (hGranzyme B) was determined by SPR. All experiments were performed at 25° C. using the Biacore 8k+ instrument (Cytiva, Marlborough, MA). The Biacore 8k+ Insight Evaluation Software Version 3.0 was used to fit the data with a 1:1 binding model to determine the association rate constant kₐ (M⁻¹s⁻¹, where “M” equals molar and “s” equals seconds) and the dissociation rate constant k_{d} (s⁻¹). These rate constants were used to calculate the equilibrium dissociation constant, K_{D} (M)=k_{d}/kₐ. To measure the binding kinetics of peptides of interest, surfaces were prepared by a capture method to a Series S Streptavidin (SA) chip using a 1×HBS-EP+ running buffer (Cytiva). hGranzyme B was diluted to 10 μg/mL in 1×HBS-EP+ and flowed over spot 2 of each flow cell on the chip at a flow rate of 10 μl/min for 20 s to achieve a level of capture of ~1100 RU. An unmodified spot 1 of each flow cell was used as a matrix binding control or reference surface. Binding kinetics were measured using single cycle kinetics at 25° C. by injecting 5 concentrations with 3-fold serial dilutions of test peptides from 0.01 μM to 1 μM. Compound dilutions were performed manually in a running buffer containing 1×HBS-EP+(Cytiva), and a final concentration of 2% DMSO. The interaction analyses over hGranzyme B, were performed using a 120 s association time and a 900 s dissociation time. All experiments were conducted at a flow rate of 50 L/min with a data collection rate of 10 Hz.

### Surface Plasmon Resonance Data Analysis

Peptide dilution series included 2 zero concentration points (no compound) which were averaged and subtracted from the peptide concentration injection as DMSO control responses. Binding data acquired from the reference surface (matrix binding control) were subtracted from the binding data for hGranzyme B surface on the chip to determine specific binding. Next, the association phase data were solvent corrected to account for any signal fluctuation throughout the experiment due to subtle DMSO concentration differences. Corrected response data were analyzed using the Biacore 8k+ Evaluation Software Version 3.0 according to the manufacturer. SPR data for representative compounds of the disclosure are found in Table 8.

TABLE 8

| | Observed m/z | Granzyme B SPR |
|---|---|---|
| Compound # | (M + 2H/2) | Kd (nM) |
| 1a | 1059.6 | 0.5 |
| 2a | 1152.0 | 1.1 |
| 3a | 1377.1 | 1.0 |
| 4a | 1179.7 | 1.1 |
| 8a | 1363.3 | 3.7 |
| 9a | 1107.1 | 188 |
| 10a | 1077.8 | 1.0 |
| 11a | 1141.7 | 2.1 |
| 12a | 1123.7 | 2.6 |
| 13a | 1187.9 | 0.1 |
| 14a | 1026.2 | 0.8 |
| 15a | 1014.3 | 0.8 |
| 16a | 1046.2 | 4.7 |
| 17a | 1045.6 | 10.7 |
| 18a | 1014.1 | 68 |
| 19a | 1022.4 | 165.0 |
| 20a | 1046.3 | 288.0 |
| 21a | 1014.3 | 453.0 |
| 22a | 1038.2 | 548.0 |
| 23a | 1014.5 | 752.0 |
| 24a | 1021.7 | 5.4 |
| 25a | 1021.8 | 199.4 |
| 26a | NA | NA |
| 27a | NA | NA |

NA = not available

### Granzyme B Biochemical Inhibition Assay

Compounds to be tested as inhibitors were manually prepared in a 2-fold serial dilutions in DMSO, spanning 200 μM to 0.39 μM in a Labcyte Echo qualified low dead volume 384-well plate. Then an ECHO acoustic liquid handler was used to dispense 125 nL of each compound from 384-well plate into a PerkinElmer Proxiplate-384 Plus F assay plate. For the positive control of inhibitor, (S)-3-((3S,6S)-3-((2S,3S)-2-acetamido-3-methylpentanamido)-4-oxo-1,2,3,4,6,7-hexahydroazepino[3,2,1-hi]indole-6-carboxamido)-4-oxobutanoic acid (see C. A. Willoughby et al./Bioorg. Med. Chem. Lett. 12 (2002) 2197-2200 2199) was used at 2 μM assay concentration. For no inhibition control, DMSO was used. Each compound was run in duplicate.

Once the compounds were dispensed in the plates, enzyme mix and substrate mix were prepared. Enzyme mix was prepared by preparing a mixture of 300 μL granzyme B (final concentration 8 nM) and 10.8 mL of assay buffer (from Sigma kit). Substrate mix was prepared by combining 500 μL substrate (from Sigma kit) and 3 mL of assay buffer (from Sigma kit). Enzyme mixture was loaded on BioRaptr and 9.25 μL were added to each well of the assay plate containing compounds. The plate was incubated at room temperature under a metal gasket cover for 30 minutes. Subsequently, 3.125 μL of substrate mix were added to each well using the BioRaptr, reaching a final assay volume of 12.5 μL. and the plate was monitored using a Pherastar FS by 1 read per minute for 30 minutes in fluorescence mode, excitation wavelength is 400 nm and emission wavelength is 510 nm.

The data analysis was performed first in excel, where the slopes of the measured fluorescence from each sample were determined using MARS software from BMG LABTECH. These rates were then multiplied by 10,000 for visualization purposes. The rates, multiplied by 10,000, were normalized to 0% and 100% activity based on the average values of the samples with either 2 μM inhibitor (S)-3-((3S,6S)-3-((2S,3S)-2-acetamido-3-methylpentanamido)-4-oxo-1,2,3,4,6,7-hexahydroazepino[3,2,1-hi]indole-6-carboxamido)-4-oxobutanoic acid, or no inhibitor respectively. All samples were then normalized against these values using the formula={100−[(sample−100% inhib)/0% inhib]}*100 to achieve a normalized percent inhibition. Normalized percent inhibition versus concentration inhibitor was then plotted in Graphpad Prism version 9.0.0 and a variable slope (four parameters) fit was calculated for each inhibitor. IC₅₀ values (see Table 9 for inhibition data for representative compounds of this disclosure) calculated by Prism to be less than 8 nM were reported as <8 nM, because the concentration of granzyme B in the assay was 8 nM.

TABLE 9
Granzyme B Inhibition Data

| Compound | Human granzyme B IC₅₀ |
|---|---|
| # | (nM) |
| 1a | 113 |

### Immunohistochemistry

To determine Granzyme B positive signal in PBMC and NOG mice, immunohistochemical (IHC) analysis of lung, liver, kidney and spleen tissue was examined. For IHC analysis, tissue was collected following in vivo PET imaging. Mice were euthanized by CO₂ and cervical dislocation in accordance with IACUC. Lung, liver, kidney and spleen tissues were then harvested and submerged in 45 mL of 10% normal buffered formalin (HistoPrep, Fisherbrand) for approximately 20-24 hr. with mild agitation at room temperature. Tissue samples were then submerged in 45 mL of 70% EtOH for processing.

Formalin fixed tissue samples were then processed using the Shandon Excelsior ES at 37° C. Briefly, the tissue samples were subjected to 70% ethanol (EtOH, HistoPrep, Fisherbrand) overnight, and then increased grades of EtOH (2×, 70% EtOH for 30 min.; 1×, 80% EtOH for 30 min.; 1×, 95% EtOH for 30 min.; 3×, 100% EtOH for 30 min.). Tissue samples were then subjected to xylene (HistoPrep, Fisherbrand) 3× for 30 min, and then paraffin (Paraplast Plus, Sigma) 3× for 30 min. Tissue samples were then immediately embedded in paraffin using a Shandon Histocentre2, and then stored at 4° C. Embedded tissue samples were sectioned at 5 μm using a HM355S automated microtome and MB35 premier microtome blades (34°/80 mm). The microtome section transfer system was set at 43° C.; the microtome section transfer system controls the water bath temperature. Tissue sections were placed onto superfrost microscope slides and allowed to air-dry at room temperature prior to IHC.

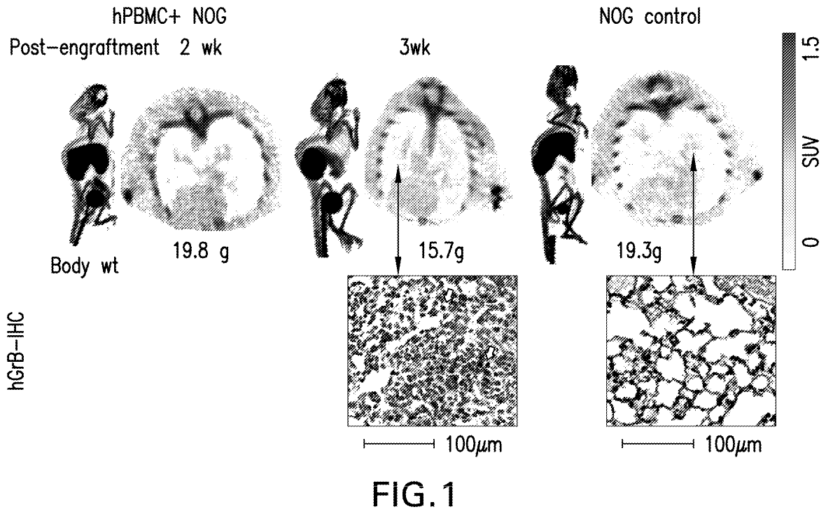

For IHC, all reagents were brought to room temperature before staining. Formalin fixed paraffin embedded (FFPE) sections were baked at 60° C. for 45 min., and immediately following deparaffinization, FFPE sections were submerged in IX Target Retrieval solution, pH 6.1 (Agilent) and subjected to heat induced epitope retrieval using a digital decloaking chamber (BioCare Medical) set at 120° C. for 4 min. at 10-15 PSI. Following epitope retrieval, FFPE sections were submerged in diH₂O and then 1× Tris Buffered Saline with Tween (TBST, 2× for 2 min., Agilent). FFPE sections were then submerged in a 3% peroxidase block (1× for 10 min., FisherScientific), washed with TBST (3× for 2 min.) and the primary antibody (MAB2906, R&D Systems) added for 60 min. at room temperature. The primary antibody was diluted (Antibody Diluent, Agilent) for a final working solution of 1 μg/mL. FFPE sections were then washed with TBST (5× for 3 min.), and the secondary antibody added (Envision Mouse Horseradish Peroxidase-HRP, Agilent) for 30 min. at room temperature. FFPE sections were then washed with TBST (5× for 3 min) and submerged in a solution of 3,3′-diaminobenzidine (DAB, Agilent). FFPE sections were then washed with diH₂O (5× for 2 min.) and submerged in Gill's Hematoxylin Solution No. 1 (Sigma), rinsed with diH₂O and submerged in Bluing Reagent S Series (Expredia). FFPE sections were then rinsed with diH₂O and baked at 60° C. until dry. FFPE sections were then cover slipped using DPX mountant for histology (Sigma). FFPE slides (see FIG. 1, lower) were scanned using an Axios slide scanner and images were then analyzed using HALO software.

### In-Vivo Preclinical Imaging

Diseased model: Graft versus host disease (GvHD) model was selected to evaluate radiolabeled granzyme-B peptides due to presence of human granzyme-B after onset of disease. Ten million human peripheral blood mononuclear cell (PBMC) were engrafted in immunocompromised NOG (NOD/Shi-scid/IL-2Rγ) mice. Body weight was monitored routinely, once the mice started to lose weight (indication of onset of disease), PET/CT scans were performed. To determine target specificity. NOG mice without human PBMC were used as control. In addition, scrambled peptide with minimal/no binding to human granzyme-B was used as additional control. Mice were intravenously injected with approximately 150 uCi (35 Ci/mmol) of ¹⁸F-labeled peptides and 45-minute whole body static PET/CT scans were performed 1 h after injection. Attenuation correction was performed using low-dose CT scan. Regions of interests (ROI) were manually drawn over tissues of interest to determine mean standardized uptake value (SUVmean).

PET imaging performed with ¹⁸F-labeled anti-granzyme-B peptide 6a from a hPBMC donor revealed significantly higher uptake in lung, liver, and bone in GvHD model than NOG control 3 week after hPBMC engraftment and onset of GvHD as observed by body weight loss (FIG. 1). No significant difference was observed at 2 weeks after hPBMC engraftment and NOG, no significant body weight loss was indicative of lack of GvHD onset.

Granzyme-B expression of infiltrating cells was confirmed by IHC performed on tissues collected from GvHD and NOG control mice. Representative human Granzyme-B IHC images at 100 μm resolution from lung tissues collected from GvHD mice (left) and NOG control mice (right) are illustrated in FIG. 1 (bottom). Granzyme-B staining is indicated by arrows.

Healthy non-human primate: A 3 h dynamic PET/CT scan was performed in a healthy male rhesus monkey to determine whole body biodistribution and clearance properties in an additional species. Approximately 6 mCi of ¹⁸F-labeled anti-granzyme-B peptide was intravenously injected in the animal. Attenuation correction was performed using low-dose CT scan. Regions of interests (ROI) were manually drawn over tissues of interest to determine mean standardized uptake value (SUVmean).

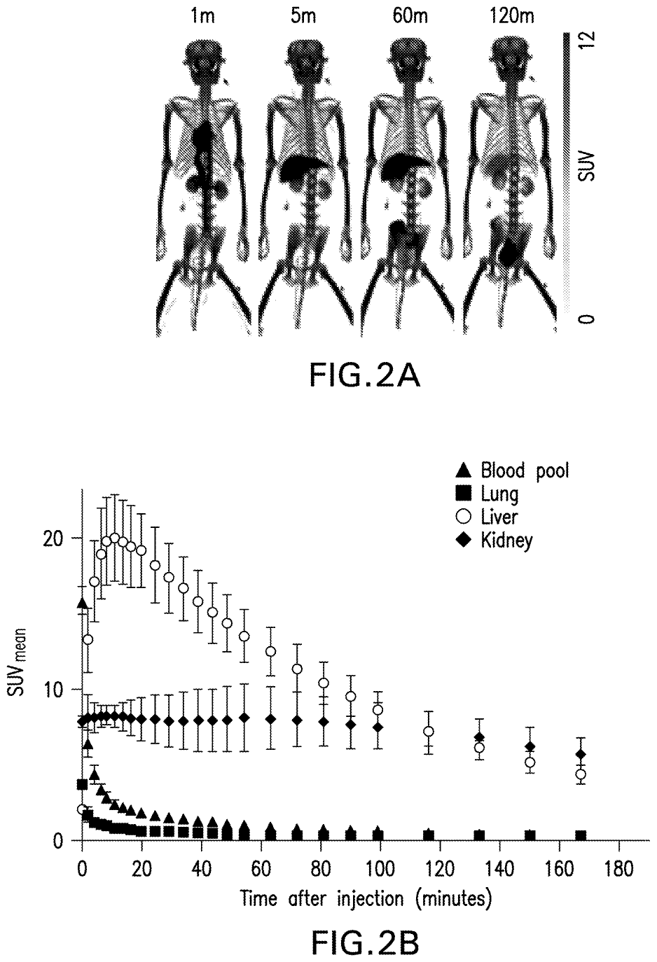

PET imaging in healthy rhesus monkeys revealed rapid blood pool clearance and high uptake in liver and kidneys within first 20 minutes of injection (FIG. 2A). 18F-labeled anti-granzyme-B peptide 6a was primarily excreted through renal route as shown in the images but has prolonged liver uptake as indicated by quantitative analyses of PET images. FIG. 2 (B) shows corresponding quantitative data presented as SUVₘₑₐₙ (right).

1. A macrocyclic peptide of Formula I: 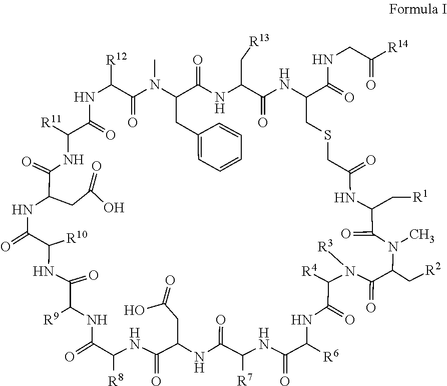
and pharmaceutically acceptable salts thereof, wherein:
R¹ and R² are each independently selected from hydrogen, C₁₋₆ alkyl and C₆₋₁₀ aryl, said aryl optionally substituted with 1 to 3 groups selected from C₁₋₆ alkyl, halogen, and hydroxyl;
R³ is hydrogen or CH₃;
R⁴, R⁶, R⁷, and R¹² are each independently selected from C₁₋₆ alkyl, and —CH₂-aryl optionally substituted with 1 to 3 substituents selected from hydroxy, C₁₋₆ alkyl and halogen,
R⁸, R⁹, R¹⁰, and R¹¹ are each independently hydrogen or C₁₋₆ alkyl, said alkyl optionally substituted with 1 to 3 groups selected from R^{a};
R^{a} is selected from hydroxyl, —COOH, —NH₂, —NHC(═NH₂)NI-2 and C(O)NH₂;
R¹³ is selected from C₃₋₁₀heteroaryl and hydrogen, said heteroaryl optionally substituted with 1 to 3 groups selected from OH, C₁₋₆ alkyl and halogen;
R¹⁴ is selected from —NH₂, —CH₂NH₂ 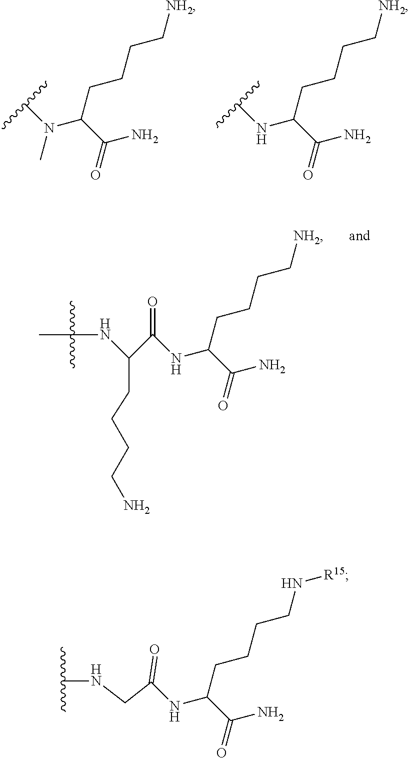
wherein R¹⁵ is selected from
(i) H;
(ii) 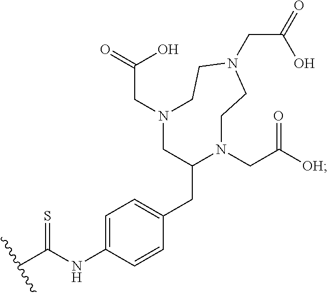 [SMILES: *C(=S)Nc1ccc(CC2CN(CC(=O)O)CCN(CC(=O)O)CCN2CC(=O)O)cc1]
(iii) R^{0a}C(O)— wherein R^{0a} is C₁₋₃ alkyl, or a polyethylene glycol polymer; and a chelating moiety, optionally having a positron emitting isotope as an imaging agent.

2. The macrocyclic peptide according to claim 1, or a pharmaceutically acceptable salt thereof, wherein R¹ is selected from C₁₋₆ alkyl and phenyl, said alkyl and phenyl optionally substituted with 1 to 3 groups selected from C₁₋₆ alkyl, halogen, and hydroxyl.

3. The macrocyclic peptide according to claim 1, or a pharmaceutically acceptable salt thereof, wherein each of R⁴, R⁶, R⁷, and R¹² are independently selected from C₁₋₆ alkyl, and (CH₂)phenyl, wherein said alkyl and phenyl are optionally substituted with 1 to 3 groups selected from C₁₋₆ alkyl, halogen, and hydroxyl.

4. The macrocyclic peptide according to claim 3, or a pharmaceutically acceptable salt thereof, wherein R⁶, R⁷, and R¹² are each (CH₂)phenyl, said phenyl optionally substituted with 1 to 3 groups selected from C₁₋₆ alkyl, halogen, and hydroxyl.

5. The macrocyclic peptide according to claim 1, or a pharmaceutically acceptable salt thereof, wherein R⁸, R⁹, R¹⁰ and R¹¹, are each C₁₋₆ alkyl, said alkyl optionally substituted with 1 to 3 R^{a}.

6. The macrocyclic peptide according to claim 5, or a pharmaceutically acceptable salt thereof, wherein R⁸, R¹⁰ and R¹¹, are independently selected from methyl, ethyl, propyl, isopropyl, butyl, pentyl, and hexyl, said methyl, ethyl, propyl, isopropyl, butyl, pentyl, and hexyl optionally substituted with 1 to 3 groups selected from R.

7. The macrocyclic peptide according to claim 5, or a pharmaceutically acceptable salt thereof, wherein R⁸, R¹⁰, and R¹¹ are each optionally substituted isopropyl.

8. The macrocyclic peptide according to claim 1, or a pharmaceutically acceptable salt thereof, wherein R⁹ is C₁₋₆ alkyl, said alkyl optionally substituted with 1 to 3 R^{a}.

9. The macrocyclic peptide according to claim 8, or a pharmaceutically acceptable salt thereof, wherein R⁹ is a methylene substituted with C(O)OH.

10. The macrocyclic peptide according to claim 1, or a pharmaceutically acceptable salt thereof, wherein one of R² and R⁴ is C₁₋₆ alkyl and the other is C₆₋₁₀ aryl or CH₂C₆₋₁₀ aryl, said aryl optionally substituted with 1 to 3 groups selected from C₁₋₆ alkyl, halogen, and hydroxyl.

11. The macrocyclic peptide according to claim 10, or a pharmaceutically acceptable salt thereof, wherein each of R² and R⁴ is phenyl or CH₂phenyl, optionally substituted with 1 to 3 groups selected from C₁₋₆ alkyl, fluorine and hydroxyl.

12. The macrocyclic peptide according to claim 1, or a pharmaceutically acceptable salt thereof, wherein R¹³ is hydrogen.

13. The macrocyclic peptide according to claim 1, or a pharmaceutically acceptable salt thereof, wherein R¹³ is C₃₋₁₀heteroaryl, said heteroaryl optionally substituted with 1 to 3 groups selected from OH, C₁₋₆ alkyl and halogen;

14. The macrocyclic peptide according to claim 13, or a pharmaceutically acceptable salt thereof, wherein R¹³ is optionally substituted imidazolyl.

15. The macrocyclic peptide according to claim 1, or a pharmaceutically acceptable salt thereof, wherein R¹⁴ is NH₂.

16. The macrocyclic peptide according to claim 1, or a pharmaceutically acceptable salt Thereof, wherein R¹⁴ is

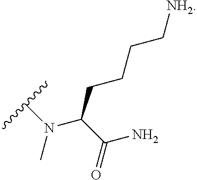 [SMILES: *N(C)[C@@H](CCCCN)C(N)=O]

17. The macrocyclic peptide according to claim 1, or a pharmaceutically acceptable salt thereof, wherein R¹⁴ is

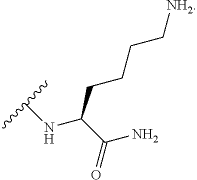 [SMILES: *N[C@@H](CCCCN)C(N)=O]

18. The macrocyclic peptide according to claim 1, or a pharmaceutically acceptable salt thereof, wherein R¹⁴ is

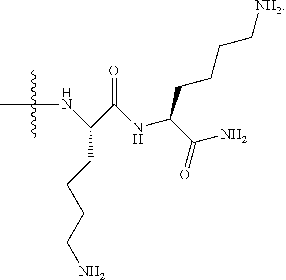 [SMILES: *N[C@@H](CCCCN)C(=O)N[C@@H](CCCCN)C(N)=O]

19. The macrocyclic peptide according to claim 1, or a pharmaceutically acceptable salt thereof, wherein R¹⁴ is

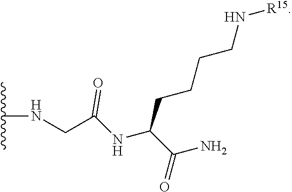

20. The macrocyclic peptide according to claim 19, or a pharmaceutically acceptable salt thereof, wherein R¹⁵ is independently selected from hydrogen and R^{0a}C(O)— wherein R^{0a} is C₁₋₃ alkyl, or a polyethylene glycol polymer selected from PEG 1-24.

21. The macrocyclic peptide according to claim 19 or a pharmaceutically acceptable salt thereof, wherein R¹⁵ is a chelating moiety independently selected from desferrioxamine; 1, 4, 7, 10-tetraacetic acid; diethylenetriaminepenaacetic acid; ethylenediaminetetraacetic acid; (1, 4, 7, 10-Tetraazacyclododecane-1, 4, 7, 10-tetra(methylene phosphonic) acid; (1R, 4R, 7R, 10R)-α′α″α′″-tetramethyl-1, 4, 7, 10-tetraazacyclododecane 1, 4, 7, 10-tetraacetic acid; 1, 4, 8, 11-Tetraazacyclotetradecane-1, 4, 8, 11-tetraacetic acid; H₄octapa, H₆phospa, H2dedpa, H₅decapa, H2azapa; HOPO; DO2 Å; 1, 4, 7, 10-Tetrakis(carbamoylmethyl)-1, 4, 7, 10-tetraazacyclododecane; 1, 4, 7-triazacyclononane-N, N′,N″-triacetic acid; 1, 4, 8, 11-tetraazabicyclo[6.6.2]hexadecane-4, 11-dicetic acid; 1, 4, 7, 10-Tetraazacyclododecane; 1, 4, 8, 11-tetraazacyclotetradecane, octadentate chelator, hexadentate chelator, phosphonate-based chelator, macrocyclic chelator, chelators comprising macrocyclic terephthalamide ligands, bifunctional chelator, fusarinine C and fusarinine C derivative chelator, triacetylfusarinine C, ferrioxamine E, ferrioxamine B, and ferrichrome A, said chelating moiety optionally having a positron emitting isotope as an imaging agent.

22. The macrocyclic peptide according to claim 19, or a pharmaceutically acceptable salt thereof, wherein R¹⁵ is selected from 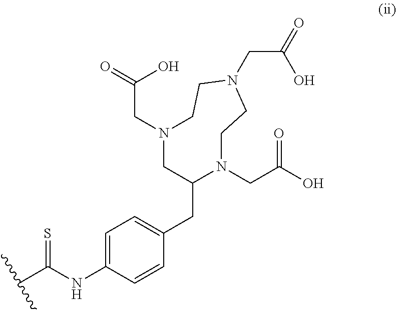 [SMILES: *C(=S)Nc1ccc(CC2CN(CC(=O)O)CCN(CC(=O)O)CCN2CC(=O)O)cc1]
wherein (ii) is optionally chelated to a positron emitting isotope.

23. The macrocyclic peptide or a pharmaceutically acceptable salt thereof according to claim 22 wherein the positron emitting isotope is selected from ⁶⁷Ga, ⁶⁸Ga, ¹⁸F, ⁶⁴Cu, ¹¹¹In and ¹⁸F—Al.

24. The macrocyclic peptide or a pharmaceutically acceptable salt thereof according to claim 1, which comprises a chelating moiety.

25. The macrocyclic peptide or a pharmaceutically acceptable salt thereof according to claim 24 wherein the chelating moiety comprises a positron emitting isotope is selected from ⁶⁸Ga, ¹⁸F, and ⁶⁴Cu.

26. The macrocyclic peptide according to claim 1, or a pharmaceutically acceptable salt thereof, represented by structural Formula I′: 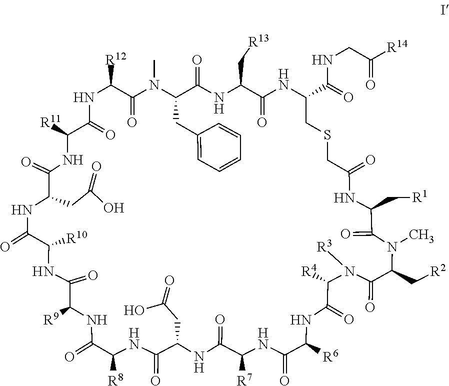
said Formula I′ optionally containing 1 or more positron emitting imaging agents.

27. A compound or pharmaceutically acceptable salt thereof, selected from

| Compd | |
|---|---|
| # | Linear Peptide Sequence |
| 1 | ClAc.F.NMeF.NMeF.Y.Y.D.V.D.V.D.V.Y.NMeF.H.C.G-NH₂ (SEQ ID NO: 8) |
| 2 | ClAc.F.NMeF.NMeF.Y.Y.D.V.D.V.D.V.Y.NMeF.H.C.G.G.K-NH₂ (SEQ ID NO: |
| | 9) |
| 3 | ClAc.F.NMeF.NMeF.Y.Y.D.V.D.V.D.V.Y.NMeF.H.C.G.G.K(NOTA)-NH₂ (SEQ |
| | ID NO: 34) |
| 4 | ClAc.F.NMeF.NMeF.Y.Y.D.V.D.V.D.V.Y.NMeF.H.C.G.G.K(Prop)-NH₂ (SEQ ID |
| | NO: 35) |
| 5 | ClAc.F.NMeF.NMeF.Y.Y.D.V.D.V.D.V.Y.NMeF.H.C.G.G.K(⁶⁸Ga-NOTA)-NH₂ |
| | (SEQ ID NO: 36) |
| 6 | ClAc.F.NMeF.NMeF.Y.Y.D.V.D.V.D.V.Y.NMeF.H.C.G.G.K(¹⁸F-AlF-NOTA)- |
| | NH₂ (SEQ ID NO: 37) |
| 7 | ClAc.F.NMeF.NMeF.Y.Y.D.V.D.A.D.V.Y.NMeF.H.C.G.G.K(¹⁸F-AlF-NOTA)- |
| | NH₂ (SEQ ID NO: 38) |
| 8 | ClAc.F.NMeF.NMeF.Y.Y.D.V.D.A.D.V.Y.NMeF.H.C.G.G.K(NOTA)-NH₂ (SEQ |
| | ID NO: 39) |
| 9 | ClAc.F.NMeF.A.Y.Y.D.V.D.V.D.V.Y.NMeF.H.C.G.G.K-NH₂ (SEQ ID NO: 40) |
| 10 | ClAc.F.NMeF.NMeF.Y.A.D.V.D.V.D.V.Y.NMeF.H.C.G.K-NH₂ (SEQ ID NO: 41) |
| 11 | ClAc.F.NMeF.NMeF.Y.A.D.V.D.V.D.V.Y.NMeF.H.C.G.K.K-NH₂ (SEQ ID NO: |
| | 42) |
| 12 | ClAc.F.NMeF.NMeF.Y.Y.D.V.D.V.D.V.Y.NMeF.H.C.G.K-NH₂ (SEQ ID NO: 43) |
| 13 | ClAc.F.NMeF.NMeF.Y.Y.D.V.D.V.D.V.Y.NMeF.H.C.G.K.K-NH₂ (SEQ ID NO: |
| | 44) |
| 14 | ClAc.F.NMeF.NMeF.Y.Y.D.V.D.V.D.V.Y.NMeF.A.C.G-NH₂ (SEQ ID NO: 45) |
| 15 | ClAc.F.NMeF.NMeF.Y.A.D.V.D.V.D.V.Y.NMeF.H.C.G-NH₂ (SEQ ID NO: 46) |
| 16 | ClAc.F.NMeF.NMeF.Y.Y.D.V.D.A.D.V.Y.NMeF.H.C.G-NH₂ (SEQ ID NO: 47) |
| 17 | ClAc.F.NMeF.NMeF.Y.Y.D.V.D.V.D.A.Y.NMeF.H.C.G-NH₂ (SEQ ID NO: 48) |
| 18 | ClAc.F.NMeF.A.Y.Y.D.V.D.V.D.V.Y.NMeF.H.C.G-NH₂ (SEQ ID NO: 49) |
| 19 | CIAc.A.NMeF.NMeF.Y.Y.D.V.D.V.D.V.Y.NMeF.H.C.G-NH₂ (SEQ ID NO: 50) |
| 20 | ClAc.F.NMeF.NMeF.Y.Y.D.A.D.V.D.V.Y.NMeF.H.C.G-NH₂ (SEQ ID NO: 51) |
| 21 | ClAc.F.NMeF.NMeF.Y.Y.D.V.D.V.D.V.A.NMeF.H.C.G-NH₂ (SEQ ID NO: 52) |
| 22 | ClAc.F.NMeF.NMeF.Y.Y.D.V.A.V.D.V.Y.NMeF.H.C.G-NH₂ (SEQ ID NO: 53) |
| 23 | ClAc.F.NMeF.NMeF.A.Y.D.V.D.V.D.V.Y.NMeF.H.C.G-NH₂ (SEQ ID NO: 54) |
| 24 | ClAc.F.NMeF.NMeA.Y.Y.D.V.D.V.D.V.Y.NMeF.H.C.G-NH₂ (SEQ ID NO: 55) |
| 25 | ClAc.F.NMeA.NMeF.Y.Y.D.V.D.V.D.V.Y.NMeF.H.C.G-NH₂ (SEQ ID NO: 16) |
| 26 | ClAc.F.NMeF.NMeF.Y.Y.D.V.D.V.D.V.Y.NMeF.H.C.G.G.K(¹¹¹In-NOTA)-NH₂ |
| | (SEQ ID NO: 18) |
| 27 | ClAc.F.NMeF.NMeF.Y.Y.D.V.D.V.D.V.Y.NMeF.H.C.G.G.K(⁶⁷Ga-NOTA)-NH₂ |
| | (SEQ ID NO: 10) |

28. A macrocyclic peptide, or pharmaceutically acceptable salt thereof of claim 1, selected from:

| Compd # | Macrocyclic Peptide Sequence |
|---|---|
| 1a | *F.NMeF.NMeF.Y.Y.D.V.D.V.D.V.Y.NMeF.H.*C.G-NH₂ (SEQ ID NO: 2) |
| 2a | *F.NMeF.NMeF.Y.Y.D.V.D.V.D.V.Y.NMeF.H.*C.G.G.K-NH₂ (SEQ ID NO: |
| | 3) |
| 3a | *F.NMeF.NmeF.Y.Y.D.V.D.V.D.V.Y.NmeF.H.*C.G.G.K(NOTA)-NH₂ (SEQ |
| | ID NO: 4) |
| 4a | *F.NMeF.NMeF.Y.Y.D.V.D.V.D.V.Y.NMeF.H.*C.G.G.K(Prop)-NH₂ (SEQ ID |
| | NO: 5) |
| 5a | *F.NMeF.NMeF.Y.Y.D.V.D.V.D.V.Y.NMeF.H.*C.G.G.K(⁶⁸Ga-NOTA)-NH₂ |
| | (SEQ ID NO: 6) |
| 6a | *F.NMeF.NMeF.Y.Y.D.V.D.V.D.V.Y.NMeF.H.*C.G.G.K(¹⁸F-AIF-NOTA)- |
| | NH₂ (SEQ ID NO: 7) |
| 7a | *F.NMeF.NMeF.Y.Y.D.V.D.A.D.V.Y.NMeF.H.*C.G.G.K(¹⁸F-AIF-NOTA)- |
| | NH₂ (SEQ ID NO: 19) |
| 8a | *F.NMeF.NMeF.Y.Y.D.V.D.A.D.V.Y.NMeF.H.*C.G.G.K(NOTA)-NH₂ (SEQ |
| | ID NO: 17) |
| 9a | *F.NMeF.A.Y.Y.D.V.D.V.D.V.Y.NMeF.H.*C.G.G.K-NH₂ (SEQ ID NO: 11) |
| 10a | *F.NMeF.NMeF.Y.A.D.V.D.V.D.V.Y.NMeF.H.*C.G.K-NH₂ (SEQ ID NO: 12) |
| 11a | *F.NMeF.NMeF.Y.A.D.V.D.V.D.V.Y.NMeF.H.*C.G.K.K-NH₂ (SEQ ID NO: |
| | 13) |
| 12a | *F.NMeF.NMeF.Y.Y.D.V.D.V.D.V.Y.NMeF.H.*C.G.K-NH₂ (SEQ ID NO: 14) |
| 13a | *F.NMeF.NMeF.Y.Y.D.V.D.V.D.V.Y.NMeF.H.*C.G.K.K-NH₂ (SEQ ID NO: |
| | 15) |
| 14a | *F.NMeF.NMeF.Y.Y.D.V.D.V.D.V.Y.NMeF.A.*C.G-NH₂ (SEQ ID NO: 22) |
| 15a | *F.NMeF.NMeF.Y.A.D.V.D.V.D.V.Y.NMeF.H.*C.G-NH₂ (SEQ ID NO: 23) |
| 16a | *F.NMeF.NMeF.Y.Y.D.V.D.A.D.V.Y.NMeF.H.*C.G-NH₂ (SEQ ID NO: 24) |
| 17a | *F.NMeF.NMeF.Y.Y.D.V.D.V.D.A.Y.NMeF.H.*C.G-NH₂ (SEQ ID NO: 25) |
| 18a | *F.NMeF.A.Y.Y.D.V.D.V.D.V.Y.NMeF.H.*C.G-NH₂ (SEQ ID NO: 26) |
| 19a | *A.NMeF.NMeF.Y.Y.D.V.D.V.D.V.Y.NMeF.H.*C.G-NH₂ (SEQ ID NO: 27) |
| 20a | *F.NMeF.NMeF.Y.Y.D.A.D.V.D.V.Y.NMeF.H.*C.G-NH₂ (SEQ ID NO: 28) |
| 21a | *F.NMeF.NMeF.Y.Y.D.V.D.V.D.V.A.NMeF.H.*C.G-NH₂ (SEQ ID NO: 29) |
| 22a | *F.NMeF.NMeF.Y.Y.D.V.A.V.D.V.Y.NMeF.H.*C.G-NH₂ (SEQ ID NO: 30) |
| 23a | *F.NMeF.NMeF.A.Y.D.V.D.V.D.V.Y.NMeF.H.*C.G-NH₂ (SEQ ID NO: 31) |
| 24a | *F.NMeF.NMeA.Y.Y.D.V.D.V.D.V.Y.NMeF.H.*C.G-NH₂ (SEQ ID NO: 32) |
| 25a | *F.NMeA.NMeF.Y.Y.D.V.D.V.D.V.Y.NMeF.H.*C.G-NH₂ (SEQ ID NO: 33) |
| 26a | *F.NMeF.NMeF.Y.Y.D.V.D.V.D.V.Y.NMeF.H.*C.G.G.K(¹¹¹In-NOTA)-NH₂ |
| | (SEQ ID NO: 20) |
| 27a | *F.NMeF.NMeF.Y.Y.D.V.D.V.D.V.Y.NMeF.H.*C.G.G.K(⁶⁷Ga-NOTA)-NH₂ |
| | (SEQ ID NO: 21) |

wherein * represent point of amino acid linkage between amino acid phenylalanine (F), or alanine (A) and sulfur on amino acid cysteine (C).

29. The macrocyclic peptide of claim 1, or a pharmaceutically acceptable salt thereof, which is a binder of granzyme B.

30. The macrocyclic peptide of claim 1, or a pharmaceutically acceptable salt thereof, which is an inhibitor of granzyme B.

31. A method of imaging granzyme B in a cell or tissue comprising contacting the cell or tissue with a of macrocyclic peptide of claim 1, or a pharmaceutically acceptable salt thereof and imaging the cell or tissue with a suitable imaging technique, thereby imaging granzyme B in the cell or tissue.

32. A method for treating a disease where granzyme B is implicated, comprising administering to a subject in need thereof a macrocyclic peptide of claim 1, or a pharmaceutically acceptable salt thereof.

33.-34. (canceled)

35. A pharmaceutical composition comprising a macrocyclic peptide of claim 1, and a pharmaceutically acceptable carrier or excipient.
